(19) United States
(12) Patent Application Publication
(10) Pub. No.: US 2014/0178961 A1
(43) Pub. Date: Jun. 26, 2014

(54) CONSTRUCTS AND STRAINS FOR FIXING CARBON DIOXIDE AND METHODS FOR PREPARING THE SAME

(71) Applicant: SHELL OIL COMPANY, Houston, TX (US)

(72) Inventors: Xuefeng LU, Qingdao (CN); Liu YANG, Qingdao (CN); Zhengxu GAO, Qingdao (CN); Xiaoming TAN, Qingdao (CN)

(73) Assignee: SHELL OIL COMPANY, Houston, TX (US)

(21) Appl. No.: 14/133,843

(22) Filed: Dec. 19, 2013

(30) Foreign Application Priority Data
Dec. 21, 2012 (CN) 201210559843.6

(51) Int. Cl. C12P 1/04

(52) U.S. Cl. CPC C12P 1/04
USPC 435/170; 435/254.21; 435/254.23; 435/254.3; 435/252.33; 435/252.31; 435/252.34; 435/252.32; 435/252.3; 435/266; 435/320.1

(57) ABSTRACT

The present invention relates to a construct for accomplishing fixation of carbon dioxide and/or reduction of carbon dioxide emission in a heterotrophic microorganism (for example, a heterotrophic fermentation strain, such as E. coli), a vector comprising the construct, a heterotrophic microorganism (for example, a heterotrophic fermentation strain, such as E. coli) comprising the construct or being transformed with the vector, and a method for fixing carbon dioxide and/or reducing carbon dioxide emission in a heterotrophic microorganism (for example, a heterotrophic fermentation strain, such as E. coli).

### CROSS-REFERENCE TO RELATED APPLICATIONS

This application claims the benefit of Chinese Application No. 201210559843.6, filed on Dec. 21, 2012, the disclosure of which is incorporated by reference herein in its entirety.

### TECHNICAL FIELD

The present disclosure generally relates to the field of biomass energy resources, the field of biochemistry and the field of genetic engineering. Specifically, the present disclosure relates to a construct for accomplishing fixation of carbon dioxide and/or reduction of carbon dioxide emission in a heterotrophic microorganism (for example, a heterotrophic fermentation strain, such as E. coli), a vector comprising the construct, a heterotrophic microorganism (for example, a heterotrophic fermentation strain, such as E. coli) comprising the construct or being transformed with the vector, and a method for fixing carbon dioxide and/or reducing carbon dioxide emission in a heterotrophic microorganism (for example, a heterotrophic fermentation strain, such as E. coli).

### BACKGROUND

This section is intended to introduce various aspects of the art, which may be associated with exemplary embodiments of the present invention. This discussion is believed to assist in providing a framework to facilitate a better understanding of particular aspects of the present invention. Accordingly, it should be understood that this section should be read in this light, and not necessarily as admissions of any prior art.

Due to sustaining high-speed increase of energy needs, rising energy price, undersupply of fossil energy as well as the problems caused by excessive use of fossil energy such as environmental pollution and climate change, the development of renewable alternative energy resources is extremely urgent. As to the problems such as reducing dependence on petroleum resources and reducing carbon dioxide emission, the techniques for producing biofuel and biochemical products, which are based on employment of biomass resources, are attractive and effective means. However, carbon dioxide emission, resulted from the production of biofuel and biochemical products, makes environment-friendly characteristic and “zero carbon emission” of bioproducts contentious. During fermentation of biomass, due to the metabolism of microorganisms, carbon dioxide emission is essentially unavoidable. The degradation and oxidation of most of active and functional biomacromolecules such as carbohydrates, lipids and proteins are accompanied by carbon dioxide generation and/or emission. For example, when glucoses are used as substrate in anaerobic fermentation of biomass to produce bioproducts and derivatize saccharides, the generation of each ethanol molecule is accompanied by the emission of one carbon dioxide molecule: C₆H₁₂O₆→2C₂H₅OH+2 CO₂.

Therefore, no matter in view of scientific study or optimization of industrial production and environmental protection, it is quite necessary to reduce or avoid carbon dioxide emission during fermentation of microorganisms. As evidenced by the foregoing, it is desirable to reduce or avoid carbon dioxide emission during fermentation of microorganisms.

### SUMMARY

The present disclosure generally relates to the field of biomass energy resources, the field of biochemistry and the field of genetic engineering. Specifically, the present disclosure relates to accomplishing fixation of carbon dioxide and/or reduction of carbon dioxide emission in a heterotrophic microorganism.

In plants and autotrophic microorganisms, carbon dioxide may be fixed and converted into organic biomass. Currently, six carbon dioxide fixation pathways have been identified, including, for example, Calvin cycle, Ribolose-Monophosphate Pathway, Serine Pathway, etc. (see, for example, FIG. 1B). These metabolic pathways are present in different biological systems, and reducing power is provided by various energies such as light, sulfide and hydrogen under anaerobic or aerobic environment.

In the present application, the inventors creatively introduce a prokaryotic CO₂ fixation pathway into a heterotrophic microorganism, which substantially reduces carbon dioxide emission/release during fermentation of the microorganism (see, for example, FIG. 1C) and therefore provides a new means for solving the problem of carbon dioxide emission during fermentation of microorganisms.

According to one aspect, there is provided a construct comprising: a first gene and a second gene, wherein the first gene is selected from the group consisting of: 1) phosphoribulokinase (Prk) genes (EC2.7.1.19); 2) genes, the nucleotide sequences of which have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 1), and which encode a protein having phosphoribulokinase activity; and 3) genes, the nucleotide sequences of which are capable of hybridizing with the sequences of the genes listed in 1) under stringent hybridization conditions, preferably highly stringent hybridization conditions, and which encode a protein having phosphoribulokinase activity; and wherein the second gene is selected from the group consisting of: 4) Ribulose-1,5-bisphosphate carboxylase/oxygenase (Rubisco) genes (EC 4.1.1.39); 5) genes, the nucleotide sequences of which have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 4), and which encode a protein having Ribulose-1,5-bisphosphate/oxygenase activity; and 6) genes, the nucleotide sequences of which are capable of hybridizing with the sequences of the genes listed in 4) under stringent hybridization conditions, preferably highly stringent hybridization conditions, and which encode a protein having Ribulose-1,5-bisphosphate/oxygenase activity.

In one embodiment, the construct further comprises an expression regulatory sequence operably linked to the first gene and/or the second gene, such as a promoter, a terminator and/or an enhancer. In one embodiment, the promoter is a constitutive promoter or an inducible promoter; and preferably, the promoter is selected from the group consisting of T7 promoter, CMV promoter, pBAD promoter, Trc promoter, Tac promoter and lacUV5 promoter; more preferably, the promoter is T7 promoter. In one embodiment, the phosphoribulokinase (Prk) genes are those derived from cyanobacteria (such as Anabaena, Synechococcus or Synechocystis) or chlorella (such as, Prochlorococcus); for example, the phosphoribulokinase (Prk) gene encodes a protein as shown in SEQ ID NO: 7; for example, the phosphoribulokinase (Prk) gene has the sequence as shown in SEQ ID NO: 1.

In one embodiment, the Ribulose-1,5-bisphosphate carboxylase/oxygenase (Rubisco) genes are those derived from cyanobacteria (such as Anabaena, Synechococcus or Synechocystis) or chlorella (such as, Prochlorococcus) or plants (such as Arabidopsis thaliana); for example, the Ribulose-1,5-bisphosphate carboxylase/oxygenase (Rubisco) gene encodes three subunits as shown in SEQ ID NOs: 8-10; for example, the Ribulose-1,5-bisphosphate carboxylase/oxygenase (Rubisco) gene has the sequence as shown in SEQ ID NO: 2. In one embodiment, the construct further comprises a marker gene for screening transformants; and preferably, the marker gene is kanamycin resistance gene, erythromycin resistance gene or spectinomycin resistance gene. In one embodiment, there is provided a vector comprising a construct described herein.

In one embodiment, there is provided a host comprising the construct of a construct and/or vector described herein. In one embodiment, the host is a heterotrophic microorganism, such as heterotrophic bacteria, fungus, and yeast, such as Saccharomyces cerevisiae, Pichia, Aspergillus niger, E. coli, Bacillus aceticus, Pseudomonas, Bacillus brevis, Corynebacterium, Bacillus subtilis, Bacillus stearothermophilus, Clostridium acetobutylicum, Clostridium butyricum, Clostridium pasteurianum; preferably, E. coli. In one embodiment, the host is E. coli as deposited in China General Microbiological Culture Collection Center (CGMCC) under Accession Number of CGMCC No. 5435.

In one embodiment, there is provided a combination comprising a first construct comprising a first gene and a second construct comprising a second gene, wherein the first gene is selected from the group consisting of: 1) phosphoribulokinase (Prk) genes (EC2.7.1.19); 2) genes, the nucleotide sequences of which have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 1), and which encode a protein having phosphoribulokinase activity; and 3) genes, the nucleotide sequences of which are capable of hybridizing with the sequences of the genes listed in 1) under stringent hybridization conditions, preferably highly stringent hybridization conditions, and which encode a protein having phosphoribulokinase activity; and wherein the second gene is selected from the group consisting of: 4) Ribulose-1,5-bisphosphate carboxylase/oxygenase (Rubisco) genes (EC 4.1.1.39); 5) genes, the nucleotide sequences of which have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 4), and which encode a protein having Ribulose-1,5-bisphosphate carboxylase/oxygenase activity; and 6) genes, the nucleotide sequences of which are capable of hybridizing with the sequences of the genes listed in 4) under stringent hybridization conditions, preferably highly stringent hybridization conditions, and which encode a protein having Ribulose-1,5-bisphosphate carboxylase/oxygenase activity.

In one embodiment, the first construct and the second construct are present as separated components, or present as a mixture of them. In one embodiment, the first construct further comprises an expression regulatory sequence operably linked to the first gene, and/or, the second construct further comprises an expression regulatory sequence operably linked to the second gene; for example, the expression regulatory sequence is selected from the group consisting of a promoter, a terminator and/or an enhancer. In one embodiment, the promoter is a constitutive promoter or an inducible promoter; and preferably, the promoter is selected from the group consisting of T7 promoter, CMV promoter, pBAD promoter, Trc promoter, Tac promoter and lacUV5 promoter; more preferably, the promoter is T7 promoter. In one embodiment, the phosphoribulokinase (Prk) genes are those derived from cyanobacteria (such as Anabaena, Synechococcus or Synechocystis) or chlorella (such as, Prochlorococcus); for example, the phosphoribulokinase (Prk) gene encodes a protein as shown in SEQ ID NO: 7; for example, the phosphoribulokinase (Prk) gene has the sequence as shown in SEQ ID NO: 1.

In one embodiment, the Ribulose-1,5-bisphosphate carboxylase/oxygenase (Rubisco) genes are those derived from cyanobacteria (such as Anabaena, Synechococcus or Synechocystis) or chlorella (such as, Prochlorococcus) or plants (such as Arabidopsis thaliana); for example, the Ribulose-1,5-bisphosphate carboxylase/oxygenase (Rubisco) gene encodes three subunits as shown in SEQ ID NOs: 8-10; for example, the Ribulose-1,5-bisphosphate carboxylase/oxygenase (Rubisco) gene has the sequence as shown in SEQ ID NO: 2. In one embodiment, the first construct and/or the second construct further comprise a marker gene for screening transformants; and preferably, the marker gene is kanamycin resistance gene, erythromycin resistance gene or spectinomycin resistance gene.

According to another aspect, there is provided a method for fixing carbon dioxide in a heterotrophic microorganism or reducing carbon dioxide emission in a heterotrophic microorganism, comprising: introducing a first gene and a second gene into the heterotrophic microorganism, wherein the first gene is selected from the group consisting of: 1) phosphoribulokinase (Prk) genes (EC2.7.1.19); 2) genes, the nucleotide sequences of which have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 1), and which encode a protein having phosphoribulokinase activity; and 3) genes, the nucleotide sequences of which are capable of hybridizing with the sequences of the genes listed in 1) under stringent hybridization conditions, preferably highly stringent hybridization conditions, and which encode a protein having phosphoribulokinase activity; and wherein the second gene is selected from the group consisting of: 4) Ribulose-1,5-bisphosphate carboxylase/oxygenase (Rubisco) genes (EC 4.1.1.39); 5) genes, the nucleotide sequences of which have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 4), and which encode a protein having Ribulose-1,5-bisphosphate carboxylase/oxygenase activity; and 6) genes, the nucleotide sequences of which are capable of hybridizing with the sequences of the genes listed in 4) under stringent hybridization conditions, preferably highly stringent hybridization conditions, and which encode a protein having Ribulose-1,5-bisphosphate carboxylase/oxygenase activity, thereby allowing the heterotrophic microorganism to express the first gene and the second gene.

In one embodiment, the phosphoribulokinase (Prk) genes are those derived from cyanobacteria (such as Anabaena, Synechococcus or Synechocystis) or chlorella (such as, Prochlorococcus); for example, the phosphoribulokinase (Prk) gene encodes a protein as shown in SEQ ID NO: 7; for example, the phosphoribulokinase (Prk) gene has the sequence as shown in SEQ ID NO: 1. In one embodiment, the Ribulose-1,5-bisphosphate carboxylase/oxygenase (Rubisco) genes are those derived from cyanobacteria (such as Anabaena, Synechococcus or Synechocystis) or chlorella (such as, Prochlorococcus) or plants (such as Arabidopsis thaliana); for example, the Ribulose-1,5-bisphosphate carboxylase/oxygenase (Rubisco) gene encodes three subunits as shown in SEQ ID NOs: 8-10; for example, the Ribulose-1,5-bisphosphate carboxylase/oxygenase (Rubisco) gene has the sequence as shown in SEQ ID NO: 2. In one embodiment, the second gene is introduced into the heterotrophic microorganism by one or more vectors. In one embodiment, the second gene is introduced into the heterotrophic microorganism by one vector which encodes the subunits rbcL and rbcS, or the subunits rbcL, rbcS and rbcX of Ribulose-1,5-bisphosphate carboxylase/oxygenase(Rubisco); or the second gene is introduced into the heterotrophic microorganism by two vectors which encode the subunits rbcL and rbcS of Ribulose-1,5-bisphosphate carboxylase/oxygenase(Rubisco), respectively; or the second gene is introduced into the heterotrophic microorganism by three vectors, which encode the subunits rbcL, rbcS and rbcX of Ribulose-1,5-bisphosphate carboxylase/oxygenase (Rubisco), respectively. In one embodiment, the first gene and the second gene are incorporated into the genome of the heterotrophic microorganism. In one embodiment, the first gene and the second gene are present as episomes in the heterotrophic microorganism. In one embodiment, the heterotrophic microorganism is selected from the group consisting of heterotrophic bacteria, fungus, and yeast, such as Saccharomyces cerevisiae, Pichia, Aspergillus niger, E. coli, Bacillus aceticus, Pseudomonas, Bacillus brevis, Corynebacterium, Bacillus subtilis, Bacillus stearothermophilus, Clostridium acetobutylicum, Clostridium butyricum, Clostridium pasteurianum; preferably, E. coli.

According to one aspect, there is provided a kit, comprising a first component and a second component, wherein the first component comprises a vector encoding phosphoribulokinase (Prk), and the second component comprises one or more vectors encoding Ribulose-1,5-bisphosphate carboxylase/oxygenase (Rubisco), wherein, the first component and the second component are present as separated components, or present as a mixture of them. In one embodiment, the second component comprises one vector, which encodes the subunits rbcL and rbcS, or the subunits rbcL, rbcS and rbcX of Ribulose-1,5-bisphosphate carboxylase/oxygenase (Rubisco). In one embodiment, the second component comprises two vectors, which encode the subunits rbcL and rbcS of Ribulose-1,5-bisphosphate carboxylase/oxygenase (Rubisco), respectively. In one embodiment, the second component comprises three vectors, which encode the subunits rbcL, rbcS and rbcX of Ribulose-1,5-bisphosphate carboxylase/oxygenase (Rubisco), respectively.

In one embodiment, the kit further comprising an agent for introducing a vector into a host (such as a heterotrophic microorganism, such as heterotrophic bacteria, fungus, and yeast, such as Saccharomyces cerevisiae, Pichia, Aspergillus niger, E. coli, Bacillus aceticus, Pseudomonas, Bacillus brevis, Corynebacterium, Bacillus subtilis, Bacillus stearothermophilus, Clostridium acetobutylicum, Clostridium butyricum, Clostridium pasteurianum; preferably, E. coli), such as a transfection agent.

The features and advantages of the present disclosure will be readily apparent to one having ordinary skill in the art upon a reading of the description of the embodiments that follows.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one having ordinary skill in the art and the benefit of this disclosure.

FIG. 1 illustrates (A) primary metabolic pathways for producing carbon dioxide in microorganisms; (B) six pathways for fixing carbon dioxide; (C) metabolic pathways for fixing carbon dioxide established by expressing phosphoribulokinase and Ribulose-1,5-bisphosphate carboxylase/oxygenase (Rubisco) genes in a heterotrophic microorganism.

FIG. 2 illustrates the basic structure of plasmid pYL25, wherein P_{T7} represents T7 promoter, prk represents phosphoribulokinase gene, and Kan^{r} represents kanamycin resistance gene. Plasmid pYL25 was obtained by cloning prk gene (SEQ ID NO: 1) from Synechocystis sp. PCC6803 to plasmid pET28a (Novagen) using two restriction enzymes NdeI and XhoI.

FIG. 3 illustrates the basic structure of plasmid pYL33, wherein P_{T7} represents T7 promoter, Rubisco represents Ribulose-1,5-bisphosphate carboxylase/oxygenase gene, and Kan^{r} represents kanamycin resistance gene. Plasmid pYL33 was obtained by cloning rubisco gene (SEQ ID NO: 2) from Synechocystis sp. PCC6803 to plasmid pET28a (Novagen) using two restriction enzymes NdeI and XhoI.

FIG. 4 illustrates the basic structure of plasmid pYL35, wherein P_{T7} represents T7 promoter, prk represents phosphoribulokinase gene, Rubisco represents Ribulose-1,5-bisphosphate carboxylase/oxygenase gene, and Kan^{r} represents kanamycin resistance gene.

FIG. 5 shows Western Blot Assay of phosphoribulokinase and Ribulose-1,5-bisphosphate carboxylase/oxygenase, wherein prk represents phosphoribulokinase, and Rubisco represents Ribulose-1,5-bisphosphate carboxylase/oxygenase; Lanes (P+R)₁ and (P+R)₂ show the expression of Prk and Rubisco enzymes in the precipitate and supernatant obtained by sonication and centrifugation of E. coli cells transformed with plasmid pYL35, respectively; Lane Marker represents a marker for protein molecular weight. The results show that both Prk and Rubisco could be expressed in E. coli cells.

FIG. 6 shows a photo, demonstrating that both phosphoribulokinase and Ribulose-1,5-bisphosphate carboxylase/oxygenase, expressed in the transformed E. coli, have activity, wherein Prk represents the E. coli cells transformed with phosphoribulokinase gene (plasmid pYL25), Rubisco represents the E. coli cells transformed with Ribulose-1,5-bisphosphate carboxylase/oxygenase gene (plasmid pYL33), Prk+Rubisco represents the E. coli cells transformed with phosphoribulokinase and Ribulose-1,5-bisphosphate carboxylase/oxygenase genes (plasmid pYL35), and CT represents the E. coli cells transformed with the plasmid pET28a. FIG. 6A shows the growth of the transformed E. coli cells in the absence of IPTG; FIG. 6B shows the growth of the transformed E. coli cells in the presence of 0.5 mM IPTG (for inducing the expression of an exogenous gene). The results show that in the absence of IPTG, all the transformed E. coli cells grew normally; while in the presence of 0.5 mM IPTG, (1) the E. coli cells transformed with phosphoribulokinase gene (plasmid pYL25) could not grow normally, indicating that in the E. coli strain, Ribulose-5-phosphate was converted into an unmetabolizable final product, Ribulose-1,5-bisphosphate, under the catalysis of phosphoribulokinase, resulting in that the strain could not grow normally and even die, thereby demonstrating the E. coli strain could express active phosphoribulokinase; (2) the E. coli cells transformed with phosphoribulokinase and Ribulose-1,5-bisphosphate carboxylase/oxygenase genes (plasmid pYL35) could grow normally, indicating that the E. coli strain could express active Ribulose-1,5-bisphosphate carboxylase/oxygenase, which further converted the unmetabolizable Ribulose-1,5-bisphosphate into metabolizable glycerate 3-phosphate, thereby rescuing the lethal phenotype of the E. coli cells transformed with phosphoribulokinase gene (plasmid pYL25).

### DETAILED DESCRIPTION

### Relevant Terms

In the present disclosure, unless indicated otherwise, all scientific and technological terminologies used herein have the same meanings as generally understood by one skilled in the art. In addition, the laboratory procedures of cell culture, molecular genetics, nucleic acid chemistry, immunology, and analytic chemistry as used herein are all conventional procedures wildly used in the corresponding fields. Furthermore, the definitions and explanations of related terms are provided as follows for better understanding of embodiments the present invention.

As used in the present invention, the term “heterotrophic”, relative to the term “autotrophic”, has the meaning as generally understood by a person skilled in the art. In general, “autotrophic microorganisms” refer to microorganisms that can live normally without depending on any organic nutrients, and “heterotrophic microorganisms” refer to microorganisms that cannot live normally without depending on at least one organic nutrient. A typical example of autotrophic microorganisms is Cyanobacterium, such as Anabaena, Synechococcus or Synechocystis (such as Synechocystis sp. PCC6803). Cyanobacterium is a photosynthetic autotrophic prokaryotic microorganism that can fix carbon dioxide by utilizing solar energy. A typical example of heterotrophic microorganisms is Escherichia coli (E. coli), such as an E. coli strain BL21(DE3). E. coli has become a most widely-used and most representative prokaryotic heterotrophic microorganism due to the advantages such as easy culture, clear genetics, and short growth period.

As used in the present disclosure, phosphoribulokinase (prk) refers to an enzyme (EC2.7.1.19) capable of catalyzing the conversion of Ribulose-5-phosphate into Ribulose-1,5-bisphosphate in the following reaction:

ATP+Ribulose-5-phosphate [symbol] ADP+Ribulose-1,5-bisphosphate (I)

The gene encoding the wild-type phosphoribulokinase (EC2.7.1.19) is well known in the art, and is available from various public databases (such as GENBANK, EXPASY and the like). In addition, the gene encoding the wild-type phosphoribulokinase (EC2.7.1.19) may be derived from various sources, such as from cyanobacteria (such as Anabaena, Synechococcus or Synechocystis) or chlorella (such as, Prochlorococcus).

A person skilled in the art would appreciate that, mutations or variations (including, but not limited to, substitution, deletion and/or addition) may occur naturally in or be introduced artificially into a wild-type phosphoribulokinase gene or the polypeptide coding thereby, without affecting its biological function or activity (i.e. ability of catalyzing the reaction of formula I). Therefore, in the present invention, functional variants of a wild-type phosphoribulokinase gene may also be used. As used in the present invention, “functional variants of a gene” refer to variants that are different from the wild-type gene in terms of sequence, but the coding polypeptides/proteins of which still retain the function or activity of the wild-type protein. Thus, the functional variant of a wild-type phosphoribulokinase gene may be a variant, the nucleotide sequence of which has at least 80% identity, preferably at least 85% identity, more preferably at least 90% identity, more preferably at least 95% identity, such as at least 96% identity, at least 97% identity, at least 98% identity, or at least 99% identity, to the nucleotide sequence of the wild-type phosphoribulokinase gene, and which encode a protein having phosphoribulokinase activity; or may be a variant, the nucleotide sequences of which is capable of hybridizing with the nucleotide sequence of the wild-type phosphoribulokinase gene under stringent hybridizing conditions, preferably highly stringent hybridizing conditions, and which encodes a protein having phosphoribulokinase activity.

As used in the present disclosure, Ribulose-1,5-bisphosphate carboxylase/oxygenase (Rubisco) refers to an enzyme (EC 4.1.1.39) capable of catalyzing the conversion of Ribulose-1,5-bisphosphate and one molecule of carbon dioxide into two molecules of glycerate 3-phosphate in the following reaction:

Ribulose-1,5-bisphosphate+CO₂+H₂O [symbol] 2×(glycerate 3-phosphate)+2×H⁺ (II)

The gene encoding the wild-type Ribulose-1,5-bisphosphate carboxylase/oxygenase (EC 4.1.1.39) is well known in the art, and is available from various public databases (such as GENBANK, EXPASY and the like). In addition, the gene encoding the wild-type Ribulose-1,5-bisphosphate carboxylase/oxygenas (EC 4.1.1.39) may be derived from various sources, such as from cyanobacteria (such as Anabaena, Synechococcus or Synechocystis) or chlorella (such as, Prochlorococcus) or plants (such as Arabidopsis thaliana).

Generally, Ribulose-1,5-bisphosphate carboxylase/oxygenase comprise two subunits (i.e. large subunit rbcL and small subunit rbcS). However, in some organisms (for example, algae such as Synechocystis), Ribulose-1,5-bisphosphate carboxylase/oxygenase may comprise three subunits (rbcL, rbcS and rbcX).

In addition, as understood by a person skilled in the art, mutations or variations (including, but not limited to, substitution, deletion and/or addition) may occur naturally in or be introduced artificially into a wild-type Ribulose-1,5-bisphosphate carboxylase/oxygenase gene or the polypeptide coding thereby, without affecting its biological function or activity (i.e. ability of catalyzing the reaction of formula II). Therefore, in the present invention, functional variants of a wild-type Ribulose-1,5-bisphosphate carboxylase/oxygenase gene may also be used. For example, a functional variant of a wild-type Ribulose-1,5-bisphosphate carboxylase/oxygenase gene may be a variant, the nucleotide sequence of which has at least 80% identity, preferably at least 85% identity, more preferably at least 90% identity, more preferably at least 95% identity, such as at least 96% identity, at least 97% identity, at least 98% identity, or at least 99% identity, to the nucleotide sequence of the wild-type Ribulose-1,5-bisphosphate carboxylase/oxygenase gene, and which encode a protein having Ribulose-1,5-bisphosphate carboxylase/oxygenase activity; or may be a variant, the nucleotide sequences of which is capable of hybridizing with the nucleotide sequence of the wild-type Ribulose-1,5-bisphosphate carboxylase/oxygenase gene under stringent hybridizing conditions, preferably highly stringent hybridizing conditions, and which encodes a protein having Ribulose-1,5-bisphosphate carboxylase/oxygenase activity.

As used in the present invention, vector refers to a nucleic acid vehicle capable of being inserted with a DNA fragment (e.g., a gene of interest) to allow the DNA fragment (e.g., the gene of interest) being transferred to the recipient cells. When the vector allows the inserted DNA fragment being expressed, the vector is also known as an expression vector. A vector can be introduced into a host cell by transformation, transduction or transfection to express the carried DNA fragment in the host cell. The useful vectors are well known by those skilled in the art, including but not being limited to plasmids, phages, cosmids, etc.

As used in the present invention, a DNA fragment (e.g., a gene of interest) is generally operably linked to an expression regulatory sequence to carry out the constitutive or inducible expression of the DNA fragment (e.g., the gene of interest). As used in the present invention, “operably linked to” means that a molecule is linked in a way that its expected function can be achieved. For example, a gene sequence is operably linked to an expression regulatory sequence so that the expression regulatory sequence can regulate the expression of the gene sequence. As used in the present invention, “expression regulatory sequence” is a regulatory sequence required for the expression of a gene, which is well known in the art. An expression regulatory sequence usually comprises a promoter, a transcription termination sequence (i.e. a terminator), as well as other sequences such as enhancer sequence.

As used in the present invention, the term “hybridization” is intended to mean the process during which, under suitable conditions, two nucleic acid sequences bond to one another with stable and specific hydrogen bonds so as to form a double strand. These hydrogen bonds form between the complementary bases adenine (A) and thymine (T) (or uracil (U)) (this is then referred to as an A-T bond) or between the complementary bases guanine (G) and cytosine (C) (this is then referred to as a G-C bond). The hybridization of two nucleic acid sequences may be entire (reference is then made to complementary sequences), i.e. the double strand obtained during this hybridization comprises only A-T bonds and C-G bonds. The hybridization may also be partial (reference is then made to sufficiently complementary sequences), i.e. the double strand obtained comprises A-T bonds and C-G bonds allowing the double strand to form, but also bases not bonded to complementary bases. The hybridization between two complementary sequences or sufficiently complementary sequences depends on the operating conditions that are used, and in particular the stringency. The stringency is defined in particular according to the base composition of the two nucleic acid sequences, and also by the degree of mismatching between these two nucleic acid sequences. The stringency can also depend on the reaction parameters, such as the concentration and the type of ionic species present in the hybridization solution, the nature and the concentration of denaturing agents and/or the hybridization temperature. All these data are well known and the appropriate conditions can be determined by those skilled in the art.

As is known in the art, conditions for hybridizing nucleic acid sequences to each other can be described as ranging from low to high stringency. The term “stringent hybridization condition” refers to a condition, under which two nucleic acid sequences can hybridize to each other when they have an identity of at least 70%, preferably at least 80%, more preferably at least 90%; that is, a condition under which, hybridization is possible only if the double strand obtained during this hybridization comprises respectively preferably at least 70%, more preferably at least 80%, still more preferably at least 90% of A-T bonds and C-G bonds.

“Stringent hybridization condition” is well known in the art, and depends on various factors, such as the components, pH and ion strength of the buffer employed, the temperature used and the like. In particular, reference herein to hybridization conditions of low stringency includes from at least about 0% to at least about 15% v/v formamide and from at least about 1 M to at least about 2 M salt for hybridization, and from at least about 1 M to at least about 2 M salt for washing conditions. Generally, the temperature for hybridization conditions of low stringency is from about 25-30° C. to about 42° C. Reference herein to hybridization conditions of medium stringency includes from at least about 16% v/v to at least about 30% v/v formamide and from at least about 0.5 M to at least about 0.9 M salt for hybridization, and from at least about 0.5 M to at least about 0.9 M salt for washing conditions. Reference herein to hybridization conditions of high stringency includes from at least about 31% v/v to at least about 50% v/v formamide and from at least about 0.01 M to at least about 0.15 M salt for hybridization, and from at least about 0.01 M to at least about 0.15 M salt for washing conditions. In general, washing is carried out at Tₘ=69.3+0.41 (G+C) % (Marmur and Doty, 1962). However, the Tₘ of a duplex DNA decreases by 1° C. with every increase of 1% in the number of mismatch base pairs (Bonner, 1983). Formamide is optional in these hybridization conditions. Accordingly, particularly preferred stringent hybridization conditions are defined as follows: hybridization condition of low stringency is 6×SSC buffer, 1.0% w/v SDS at 25-42° C.; hybridization condition of medium stringency is 2×SSC buffer, 1.0% w/v SDS at a temperature in the range 20° C. to 65° C.; hybridization conditions of high stringency is 0.1×SSC buffer, 0.1% w/v SDS at a temperature of at least 65° C. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al, eds. (1995) Current Protocols in Molecular Biology, Chapter 2 (Greene Publishing and Wiley-Interscience, New York). Also, see Sambrook et al (1989) Molecular Cloning: A Laboratory Manual (2d ed., Cold Spring Harbor Laboratory Press, Plainview, N.Y.). The determination and selection of suitable stringent hybridization conditions are well within the ability of a person skilled in the art.

As used in the present invention, the term “identity” or “percent identity” refers to the match degree between two polypeptides or between two nucleic acids. When two sequences for comparison have the same base or amino acid monomer sub-unit at a certain site (e.g., each of two DNA molecules has an adenine at a certain site, or each of two polypeptides has a lysine at a certain site), the two molecules are identical at the site. The percent identity between two sequences is a function of the number of identical sites shared by the two sequences over the total number of sites for comparison×100. For example, if 6 of 10 sites of two sequences are matched, these two sequences have an identity of 60%. For example, DNA sequences: CTGACT and CAGGTT share an identity of 50% (3 of 6 sites are matched). Usually, the comparison of two sequences is conducted in a manner to produce maximum identity (optimal alignment). Optimal alignment can be conducted by using, for example, local homology algorithm of Smith and Waterman (Adv. Appl. Math. 2: 482, 1970); homology alignment algorithm of Needleman and Wunsch (J. Mol. Biol. 48: 443, 1970); similarity search methods of Pearson and Lipman (Proc. Natl. Acad. Sci. USA 85: 2444, 1988); computerized implementation of these algorithms (such as, GAP, BESTFIT, FASTA, BLASTP, BLASTN and TFASTA of Wisconsin Genetics Software Package, Genetics Computer Group, 575 Science Dr., Madison, Wis.); or artificial alignment and visual inspection (see, for example, Ausubel et al., Current Protocols in Molecular Biology (1995 supplementary issue)). For example, optimal alignment can be conducted by using a computer program such as Align program (DNAstar, Inc.) which is based on the method of Needleman, et al. (J. Mol. Biol. 48:443-453, 1970). The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Percent identities involved in the embodiments of the present invention include at least about 60% or at least about 65% or at least about 70% or at least about 75% or at least about 80% or at least about 85% or at least about 90% or above, such as about 95% or about 96% or about 97% or about 98% or about 99%, such as at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

The present invention is based, at least partially, on the unexpected findings of the inventors: by introducing a carbon dioxide fixation pathway (such as phosphoribulokinase and Ribulose-1,5-bisphosphate carboxylase/oxygenase) into a heterotrophic microorganism (for example, a heterotrophic fermentation strain, such as E. coli), carbon dioxide emission/release during fermentation of the heterotrophic microorganism can be reduced.

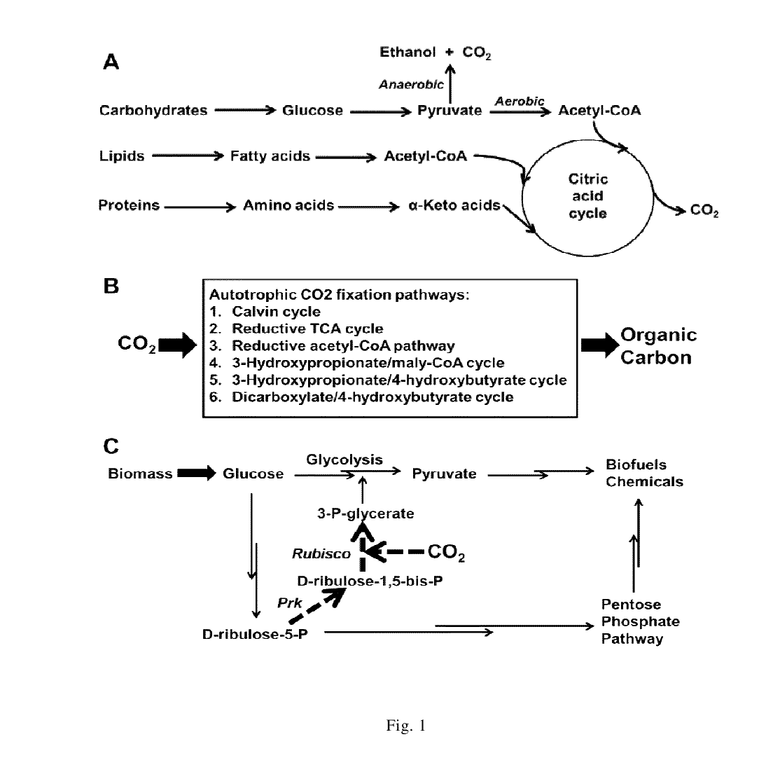

Without being limited by any theory, the inventors now believe that by introducing a carbon dioxide fixation pathway (such as phosphoribulokinase and Ribulose-1,5-bisphosphate carboxylase/oxygenase) into a heterotrophic microorganism (for example, a heterotrophic fermentation strain, such as E. coli), the heterotrophic microorganism can convert carbon dioxide to organic substances, thereby achieving fixation of carbon dioxide and/or reduction of carbon dioxide emission. For example, in case that phosphoribulokinase and Ribulose-1,5-bisphosphate carboxylase/oxygenase are introduced into a heterotrophic microorganism, the heterotrophic microorganism may utilize phosphoribulokinase to produce Ribulose-1,5-bisphosphate by using Ribulose-5-phosphate as substrate; and may further utilize Ribulose-1,5-bisphosphate carboxylase/oxygenase to produce glycerate 3-phosphate by using Ribulose-1,5-bisphosphate and carbon dioxide as substrates (see, for example, FIG. 1C), thereby converting carbon dioxide to organic substances (for example, glycerate 3-phosphate) which in turn can participate metabolism in microorganisms, and finally achieving fixation of carbon dioxide and/or lower carbon dioxide emission during fermentation of the microorganism.

Thus, in the first aspect, the present invention provides a construct, comprising a first gene and a second gene, wherein the first gene is selected from the group consisting of:

1) phosphoribulokinase (Prk) genes (EC2.7.1.19);

2) genes of which the nucleotide sequences have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 1), and which encode a protein having phosphoribulokinase activity; and

3) genes of which the nucleotide sequences are capable of hybridizing with the sequences of the genes listed in 1) under stringent hybridization conditions, preferably highly stringent hybridization conditions, and which encode a protein having phosphoribulokinase activity; and

wherein the second gene is selected from the group consisting of:

4) Ribulose-1,5-bisphosphate carboxylase/oxygenase (Rubisco) genes (EC 4.1.1.39);

5) genes, the nucleotide sequences of which have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 4), and which encode a protein having Ribulose-1,5-bisphosphate/oxygenase activity; and

6) genes, the nucleotide sequences of which are capable of hybridizing with the sequences of the genes listed in 4) under stringent hybridization conditions, preferably highly stringent hybridization conditions, and which encode a protein having Ribulose-1,5-bisphosphate/oxygenase activity.

The construct may be used to introduce a carbon dioxide fixation pathway into a heterotrophic microorganism (for example, a heterotrophic fermentation strain, such as E. coli).

In a preferred embodiment, the construct of the present invention further comprises an expression regulatory sequence operably linked to the first gene and/or the second gene, such as a promoter, a terminator and/or an enhancer. For example, the construct of the present invention further comprises an expression regulatory sequence operably linked to the first gene, and an expression regulatory sequence operably linked to the second gene.

The expression regulatory sequences are well known to a person skilled in the art. In a preferred embodiment, the promoter is a constitutive promoter or an inducible promoter. In another preferred embodiment, the promoter includes, but is not limited to, for example, T7 promoter, CMV promoter, pBAD promoter, Trc promoter, Tac promoter and lacUV5 promoter. In a further preferred embodiment, the promoter is T7 promoter.

In a preferred embodiment, after transforming a host cell with the construct, the first gene and the second gene in the construct are expressed, respectively, to produce a first protein having phosphoribulokinase activity and a second protein having Ribulose-1,5-bisphosphate carboxylase/oxygenase activity. In another preferred embodiment, the first gene and the second gene are expressed as a fusion protein in a host cell, which has phosphoribulokinase activity and Ribulose-1,5-bisphosphate carboxylase/oxygenase activity.

In a preferred embodiment, the phosphoribulokinase (Prk) genes are those derived from cyanobacteria (such as Anabaena, Synechococcus or Synechocystis) or chlorella (such as, Prochlorococcus). In a further preferred embodiment, the phosphoribulokinase (Prk) gene encodes a protein as shown in SEQ ID NO: 7; for example, the phosphoribulokinase (Prk) gene has the sequence as shown in SEQ ID NO: 1.

In a preferred embodiment, the Ribulose-1,5-bisphosphate carboxylase/oxygenase (Rubisco) genes are those derived from cyanobacteria (such as Anabaena, Synechococcus or Synechocystis) or chlorella (such as, Prochlorococcus), or plants (such as Arabidopsis thaliana). In a further preferred embodiment, the Ribulose-1,5-bisphosphate carboxylase/oxygenase (Rubisco) gene encodes three subunits as shown in SEQ ID NOs: 8-10; for example, the Ribulose-1,5-bisphosphate carboxylase/oxygenase (Rubisco) gene has the sequence as shown in SEQ ID NO: 2.

In a preferred embodiment, the construct may further comprise a marker gene for screening transformants. The marker gene includes, but is not limited to, for example, kanamycin resistance gene (NCBI ID: NC_{—}003239.1), erythromycin resistance gene (NCBI ID: NC_{—}015291.1) and spectinomycin resistance gene (see, for example, the Chinese invention patent application No. 201010213758.5). The marker genes are well known to a person skilled in the art, and the selection of them is within the ability of a person skilled in the art. In a preferred embodiment, the marker gene is kanamycin resistance gene. In another preferred embodiment, the marker gene is the Omega fragment of spectinomycin resistance gene, the sequence of which can be found in, for example, the Chinese invention patent application No. 201010213758.5. In another preferred embodiment, the marker gene may be located upstream or downstream to the promoter operably linked to the first gene and/or the second gene.

In the second aspect, the present invention provides a vector, comprising the construct as defined in the first aspect.

Vectors for inserting a gene of interest or a construct of interest are well known in the art, including, but not limited to, clonal vectors and expression vectors. In a preferred embodiment, the vector is, for example, a plasmid, cosmid, phage, and the like.

In the third aspect, the present invention provides a host, which comprises the construct and/or vector as defined above, or is transformed with the vector as defined above.

In a preferred embodiment, the host is a heterotrophic microorganism. For example, the host may be a heterotrophic bacterium, fungus, and yeast, including, but not limited to, Saccharomyces cerevisiae, Pichia, Aspergillus niger, E. coli, Bacillus aceticus, Pseudomonas, Bacillus brevis, Corynebacterium, Bacillus subtilis, Bacillus stearothermophilus, Clostridium acetobutylicum, Clostridium butyricum, Clostridium pasteurianum. Preferably, the host is E. coli.

In a preferred embodiment, the host is an E. coli E2 deposited in China General Microbiological Culture Collection Center, CGMCC, under an Accession Number of CGMCC No. 5435.

In the fourth aspect, the present invention provides a combination, comprising a first construct comprising a first gene and a second construct comprising a second gene, wherein the first gene is selected from the group consisting of:

1) phosphoribulokinase (Prk) genes (EC2.7.1.19);

2) genes, the nucleotide sequences of which have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 1), and which encode a protein having phosphoribulokinase activity; and

3) genes, the nucleotide sequences of which are capable of hybridizing with the sequences of the genes listed in 1) under stringent hybridization conditions, preferably highly stringent hybridization conditions, and which encode a protein having phosphoribulokinase activity; and

wherein the second gene is selected from the group consisting of:

4) Ribulose-1,5-bisphosphate carboxylase/oxygenase (Rubisco) genes (EC 4.1.1.39);

5) genes, the nucleotide sequences of which have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 4), and which encode a protein having Ribulose-1,5-bisphosphate carboxylase/oxygenase activity; and

6) genes, the nucleotide sequences of which are capable of hybridizing with the sequences of the genes listed in 4) under stringent hybridization conditions, preferably highly stringent hybridization conditions, and which encode a protein having Ribulose-1,5-bisphosphate carboxylase/oxygenase activity.

In a preferred embodiment, the first construct and the second construct are present as separated components, or present as a mixture of them.

In a preferred embodiment, the first construct further comprises an expression regulatory sequence operably linked to the first gene, and/or the second construct further comprises an expression regulatory sequence operably linked to the second gene, such as a promoter, a terminator and/or an enhancer.

The expression regulatory sequences are well known to a person skilled in the art. In a preferred embodiment, the promoter is a constitutive promoter or an inducible promoter. In another preferred embodiment, the promoter includes, but is not limited to, for example, T7 promoter, CMV promoter, pBAD promoter, Trc promoter, Tac promoter and lacUV5 promoter. In a further preferred embodiment, the promoter is T7 promoter.

In a preferred embodiment, the phosphoribulokinase (Prk) genes are those derived from cyanobacteria (such as Anabaena, Synechococcus or Synechocystis) or chlorella (such as, Prochlorococcus). In a further preferred embodiment, the phosphoribulokinase (Prk) gene encodes a protein as shown in SEQ ID NO: 7; for example, the phosphoribulokinase (Prk) gene has the sequence as shown in SEQ ID NO: 1.

In a preferred embodiment, the Ribulose-1,5-bisphosphate carboxylase/oxygenase (Rubisco) genes are those derived from cyanobacteria (such as Anabaena, Synechococcus or Synechocystis) or chlorella (such as, Prochlorococcus), or plants (such as Arabidopsis thaliana). In a further preferred embodiment, the Ribulose-1,5-bisphosphate carboxylase/oxygenase (Rubisco) gene encodes three subunits as shown in SEQ ID NOs: 8-10; for example, the Ribulose-1,5-bisphosphate carboxylase/oxygenase (Rubisco) gene has the sequence as shown in SEQ ID NO: 2.

In a preferred embodiment, the first construct and/or the second construct may further comprise a marker gene for screening transformants. The marker gene includes, but is not limited to, for example, kanamycin resistance gene (NCBI ID: NC_{—}003239.1), erythromycin resistance gene (NCBI ID: NC_{—}015291.1) and spectinomycin resistance gene (see, for example, the Chinese invention patent application No. 201010213758.5). The marker genes are well known to a person skilled in the art, and the selection of them is within the ability of a person skilled in the art. In a preferred embodiment, the marker gene is kanamycin resistance gene. In another preferred embodiment, the marker gene is the Omega fragment of spectinomycin resistance gene, the sequence of which can be found in, for example, the Chinese invention patent application No. 201010213758.5. In another preferred embodiment, both the first construct and the second construct comprise a marker gene. In a further preferred embodiment, the marker gene of the first construct and the marker gene of the second construct may be the same or may be different.

In the fifth aspect, the present invention provides a combination, comprising a first vector and a second vector, wherein said first vector comprises the first construct as defined in the fourth aspect, and the second vector comprises the second construct as defined in the fourth aspect.

Vectors for inserting a gene of interest or a construct of interest are well known in the art, including, but not limited to clonal vectors and expression vectors. In a preferred embodiment, the first vector and/or the second vector are independently, for example, plasmid, cosmid, phage, and the like.

In another aspect, the present invention provides a host, which comprises the first construct and/or the first vector as defined above, as well as the second construct and/or the second vector as defined above, or is transformed with the first vector and the second vector as defined above.

In a preferred embodiment, the host is a heterotrophic microorganism. For example, the host may be a heterotrophic bacterium, fungus, and yeast, including, but not limited to, Saccharomyces cerevisiae, Pichia, Aspergillus niger, E. coli, Bacillus aceticus, Pseudomonas, Bacillus brevis, Corynebacterium, Bacillus subtilis, Bacillus stearothermophilus, Clostridium acetobutylicum, Clostridium butyricum, Clostridium pasteurianum. Preferably, the host is E. coli.

In the sixth aspect, the present invention provides a kit, comprising 1) the construct as defined in the first aspect, or the vector as defined in the second aspect; and/or 2) the combination as defined in the fourth or fifth aspect.

In a preferred embodiment, the kit further comprises an additional agent, for example, an agent for introducing a construct or a vector into a host (such as a heterotrophic microorganism, such as heterotrophic bacteria, fungus, and yeast, such as Saccharomyces cerevisiae, Pichia, Aspergillus niger, E. coli, Bacillus aceticus, Pseudomonas, Bacillus brevis, Corynebacterium, Bacillus subtilis, Bacillus stearothermophilus, Clostridium acetobutylicum, Clostridium butyricum, Clostridium pasteurianum; preferably, E. coli). In a preferred embodiment, the additional agent is, for example, a transfection agent.

In the seventh aspect, the present invention provides a method for fixing CO₂ in a heterotrophic microorganism or reducing CO₂ emissions in a heterotrophic microorganism, comprising:

1) introducing the construct as defined in the first aspect, or the vector as defined in the second aspect, into the heterotrophic microorganism; or

2) introducing the first construct as defined in the fourth aspect and/or the first vector as defined in the fifth aspect, as well as the second construct as defined in the fourth aspect and/or the second vector as defined in the fifth aspect, into the heterotrophic microorganism,

thereby allowing the heterotrophic microorganism to express the first gene and the second gene.

In a preferred embodiment, the first gene and the second gene are incorporated into the genome of the heterotrophic microorganism. In another preferred embodiment, the first gene and the second gene are present as episomes in the host.

In a preferred embodiment, the host is a heterotrophic microorganism. For example, the host may be a heterotrophic bacterium, fungus, and yeast, including, but not limited to, Saccharomyces cerevisiae, Pichia, Aspergillus niger, E. coli, Bacillus aceticus, Pseudomonas, Bacillus brevis, Corynebacterium, Bacillus subtilis, Bacillus stearothermophilus, Clostridium acetobutylicum, Clostridium butyricum, Clostridium pasteurianum. Preferably, the host is E. coli.

Methods for introducing a construct or a vector into a host are well known to a person skilled in the art, including, but not limited to, transfection, transformation, and transduction. For example, the methods include, but are limited to liposome transfection, calcium phosphate deposition, electroporation, particles bombarding, and the like.

In the eighth aspect, the embodiment of the present invention relates to a use of the construct as defined in the first aspect or the vector as defined in the second aspect, or the combination as defined in the fourth or fifth aspect, or the kit as defined in the sixth aspect, for fixing carbon dioxide in a heterotrophic microorganism or for reducing carbon dioxide emission in a heterotrophic microorganism.

In a preferred embodiment, the host is a heterotrophic microorganism. For example, the host may be a heterotrophic bacterium, fungus, and yeast, including, but not limited to, Saccharomyces cerevisiae, Pichia, Aspergillus niger, E. coli, Bacillus aceticus, Pseudomonas, Bacillus brevis, Corynebacterium, Bacillus subtilis, Bacillus stearothermophilus, Clostridium acetobutylicum, Clostridium butyricum, Clostridium pasteurianum. Preferably, the host is E. coli.

In the ninth aspect, the present invention provides an E. coli strain E2 capable of fixing carbon dioxide, which was deposited in China General Microbiological Culture Collection Center, CGMCC, under an Accession Number of CGMCC No. 5435.

In the tenth aspect, the present invention provides a method for fixing carbon dioxide in a heterotrophic microorganism or reducing carbon dioxide emissions in a heterotrophic microorganism, comprising:

introducing a first gene and a second gene into the heterotrophic microorganism, wherein the first gene is selected from the group consisting of:

1) phosphoribulokinase (Prk) genes (EC2.7.1.19);

2) genes, the nucleotide sequences of which have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 1), and which encode a protein having phosphoribulokinase activity; and

3) genes, the nucleotide sequences of which are capable of hybridizing with the sequences of the genes listed in 1) under stringent hybridization conditions, preferably highly stringent hybridization conditions, and which encode a protein having phosphoribulokinase activity; and

wherein the second gene is selected from the group consisting of:

4) Ribulose-1,5-bisphosphate carboxylase/oxygenase (Rubisco) genes (EC 4.1.1.39);

5) genes, the nucleotide sequences of which have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 4), and which encode a protein having Ribulose-1,5-bisphosphate carboxylase/oxygenase activity; and

6) genes, the nucleotide sequences of which are capable of hybridizing with the sequences of the genes listed in 4) under stringent hybridization conditions, preferably highly stringent hybridization conditions, and which encode a protein having Ribulose-1,5-bisphosphate carboxylase/oxygenase activity.

thereby allowing the heterotrophic microorganism to express the first gene and the second gene.

In a preferred embodiment, the phosphoribulokinase (Prk) genes are those derived from cyanobacteria (such as Anabaena, Synechococcus or Synechocystis) or chlorella (such as, Prochlorococcus). In a further preferred embodiment, the phosphoribulokinase (Prk) gene encodes a protein as shown in SEQ ID NO: 7; for example, the phosphoribulokinase (Prk) gene has the sequence as shown in SEQ ID NO: 1.

In a preferred embodiment, the Ribulose-1,5-bisphosphate carboxylase/oxygenase (Rubisco) genes are those derived from cyanobacteria (such as Anabaena, Synechococcus or Synechocystis) or chlorella (such as, Prochlorococcus), or plants (such as Arabidopsis thaliana). In a further preferred embodiment, the Ribulose-1,5-bisphosphate carboxylase/oxygenase (Rubisco) gene encodes three subunits as shown in SEQ ID NOs: 8-10; for example, the Ribulose-1,5-bisphosphate carboxylase/oxygenase (Rubisco) gene has the sequence as shown in SEQ ID NO: 2.

The first gene and the second gene may be introduced into the heterotrophic microorganism by any method known by a person skilled in the art. Such a method includes, but is not limited to, transformation, transduction, transfection, such as liposome transfection, calcium phosphate deposition, electroporation, particles bombarding, and the like.

In addition, since Ribulose-1,5-bisphosphate carboxylase/oxygenase (Rubisco) may comprise two subunits (large subunit rbcL and small subunit rbcS) or three subunits (rbcL, rbcS and rbcX), the second gene may be introduced into the heterotrophic microorganism by one or more vectors. For example, the second gene may be introduced into the heterotrophic microorganism by one vector which encodes the subunits rbcL and rbcS (or the subunits rbcL, rbcS and rbcX) of Ribulose-1,5-bisphosphate carboxylase/oxygenase (Rubisco). Alternatively, the second gene may be introduced into the heterotrophic microorganism by two vectors, which encode a large subunit rbcL and a small subunit rbcS of Ribulose-1,5-bisphosphate carboxylase/oxygenase (Rubisco), respectively. Alternatively, the second gene may be introduced into the heterotrophic microorganism by three vectors, which encode the subunits rbcL, rbcS and rbcX of Ribulose-1,5-bisphosphate carboxylase/oxygenase (Rubisco), respectively.

In a preferred embodiment, the first gene and the second gene are incorporated into the genome of the heterotrophic microorganism. In another preferred embodiment, the first gene and the second gene are present as episomes in the host.

In a preferred embodiment, the host is a heterotrophic microorganism. For example, the host may be a heterotrophic bacterium, fungus, and yeast, including, but not limited to Saccharomyces cerevisiae, Pichia, Aspergillus niger, E. coli, Bacillus aceticus, Pseudomonas, Bacillus brevis, Corynebacterium, Bacillus subtilis, Bacillus stearothermophilus, Clostridium acetobutylicum, Clostridium butyricum, Clostridium pasteurianum. Preferably, the host is E. coli.

In the eleventh aspect, the present invention provides a kit, comprising a first component and a second component, wherein

the first component comprises a vector encoding phosphoribulokinase (Prk), and

the second component comprises one or more vectors encoding Ribulose-1,5-bisphosphate carboxylase/oxygenase (Rubisco),

wherein, the first component and the second component are present as separated components, or present as a mixture of them.

In a preferred embodiment, the second component comprises one vector, which encodes the subunits rbcL and rbcS (or the subunits rbcL, rbcS and rbcX) of Ribulose-1,5-bisphosphate carboxylase/oxygenase (Rubisco).

In another preferred embodiment, the second component comprises two vectors, which encode the subunits rbcL and rbcS of Ribulose-1,5-bisphosphate carboxylase/oxygenase (Rubisco), respectively.

In another preferred embodiment, the second component comprises three vectors, which encode the subunits rbcL, rbcS and rbcX of Ribulose-1,5-bisphosphate carboxylase/oxygenase (Rubisco), respectively.

In a preferred embodiment, the kit further comprises an additional agent, for example, an agent for introducing a construct or a vector into a host (such as a heterotrophic microorganism, such as heterotrophic bacteria, fungus, and yeast, such as Saccharomyces cerevisiae, Pichia, Aspergillus niger, E. coli, Bacillus aceticus, Pseudomonas, Bacillus brevis, Corynebacterium, Bacillus subtilis, Bacillus stearothermophilus, Clostridium acetobutylicum, Clostridium butyricum, Clostridium pasteurianum; preferably, E. coli). In a preferred embodiment, the additional agent is, for example, a transfection agent.

The present invention further provides a use of the kit as described above for fixing carbon dioxide in a heterotrophic microorganism or for reducing carbon dioxide emission in a heterotrophic microorganism.

In embodiments described herein, the inventors establish a pathway for fixing carbon dioxide in a heterotrophic microorganism (for example, a heterotrophic fermentation strain, such as E. coli) by introducing genes encoding phosphoribulokinase and Ribulose-1,5-bisphosphate carboxylase/oxygenase into the heterotrophic microorganism, thereby allowing the heterotrophic microorganism to convert carbon dioxide to organic substances, and finally achieving the fixation of carbon dioxide and reduction of carbon dioxide emission during fermentation of the heterotrophic microorganism. Therefore, one of the advantages of the embodiments of the present invention is that carbon dioxide emission is reduced during fermentation of microorganisms, so as to make the production of bioproducts and biochemical product with the microorganisms more “low carbon”. In addition, the present invention provides a new solution for the problem of carbon dioxide emission during fermentation of microorganisms, and is of important significance for optimization of industrial production and environmental protection. In particular, the embodiments of the present invention may be combined with industrial microorganisms suitable for genetic engineering to further optimize industrial production and enhance environmental friendly degree.

The embodiments of the present invention are further illustrated in detail by the following drawings and examples. However, those skilled in the art will appreciate that the drawings and examples are used only for the purpose of illustrating the present invention, rather than limiting the protection scope of the present invention. According to the following descriptions of the drawings and preferred embodiments, the objects and advantageous aspects of the present invention are apparent for those skilled in the art.

FIG. 1 illustrates (A) primary metabolic pathways for producing carbon dioxide in microorganisms; (B) six pathways for fixing carbon dioxide; (C) metabolic pathways for fixing carbon dioxide established by expressing phosphoribulokinase and Ribulose-1,5-bisphosphate carboxylase/oxygenase (Rubisco) genes in a heterotrophic microorganism.

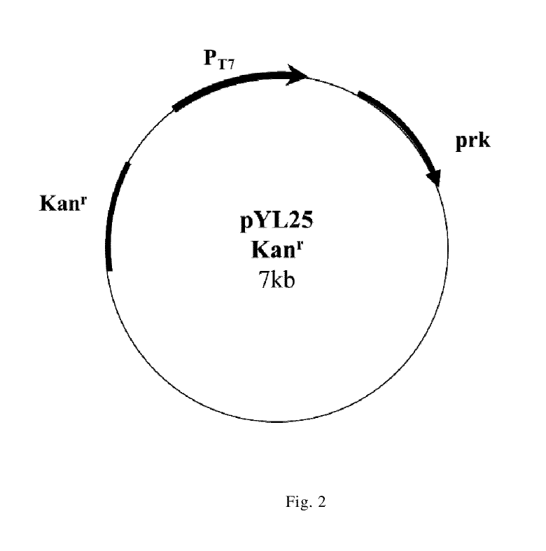

FIG. 2 illustrates the basic structure of plasmid pYL25, wherein P_{T7} represents T7 promoter, prk represents phosphoribulokinase gene, and Kan^{r} represents kanamycin resistance gene. Plasmid pYL25 was obtained by cloning prk gene (SEQ ID NO: 1) from Synechocystis sp. PCC6803 to plasmid pET28a (Novagen) using two restriction enzymes NdeI and XhoI.

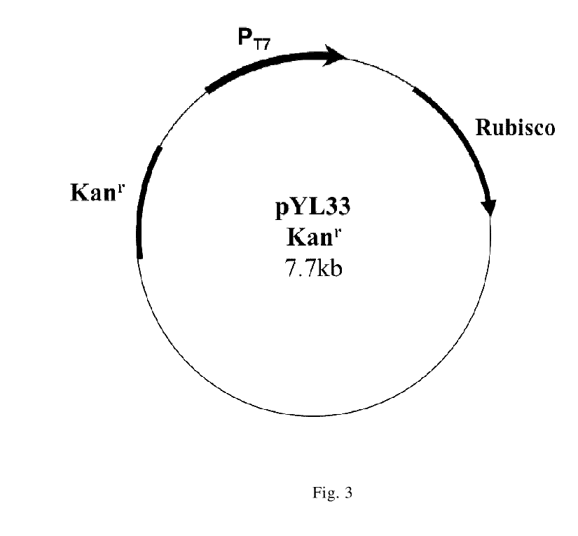

FIG. 3 illustrates the basic structure of plasmid pYL33, wherein P_{T7} represents T7 promoter, Rubisco represents Ribulose-1,5-bisphosphate carboxylase/oxygenase gene, and Kan^{r} represents kanamycin resistance gene. Plasmid pYL33 was obtained by cloning rubisco gene (SEQ ID NO: 2) from Synechocystis sp. PCC6803 to plasmid pET28a (Novagen) using two restriction enzymes NdeI and XhoI.

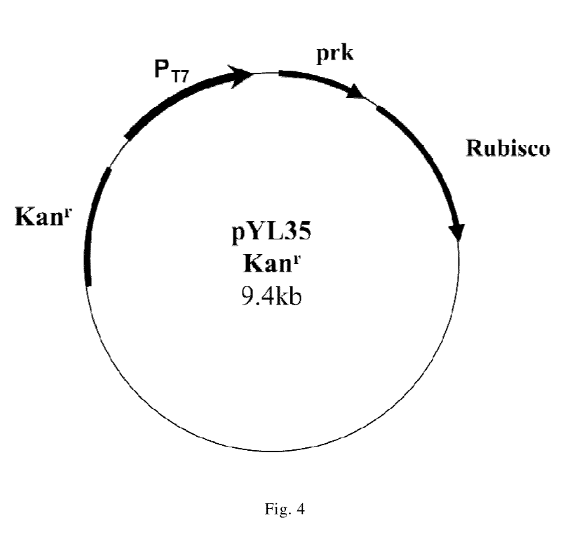

FIG. 4 illustrates the basic structure of plasmid pYL35, wherein P_{T7} represents T7 promoter, prk represents phosphoribulokinase gene, Rubisco represents Ribulose-1,5-bisphosphate carboxylase/oxygenase gene, and Kan^{r} represents kanamycin resistance gene.

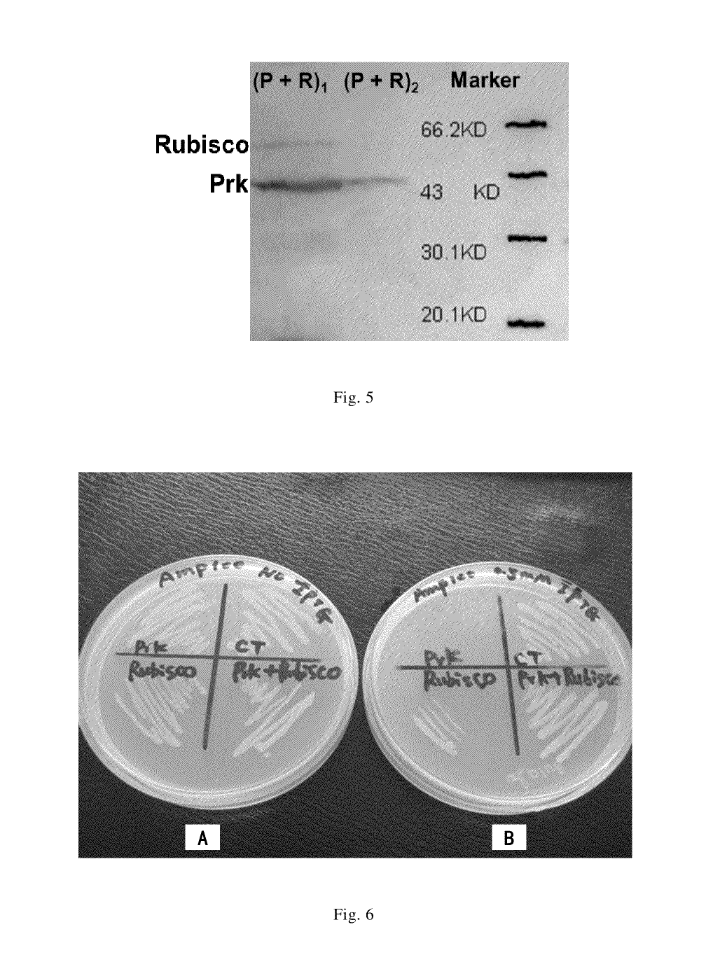

FIG. 5 shows Western Blot Assay of phosphoribulokinase and Ribulose-1,5-bisphosphate carboxylase/oxygenase, wherein prk represents phosphoribulokinase, and Rubisco represents Ribulose-1,5-bisphosphate carboxylase/oxygenase; Lanes (P+R)₁ and (P+R)₂ show the expression of Prk and Rubisco enzymes in the precipitate and supernatant obtained by sonication and centrifugation of E. coli cells transformed with plasmid pYL35, respectively; Lane Marker represents a marker for protein molecular weight. The results show that both Prk and Rubisco could be expressed in E. coli cells.

FIG. 6 shows a photo, demonstrating that both phosphoribulokinase and Ribulose-1,5-bisphosphate carboxylase/oxygenase, expressed in the transformed E. coli, have activity, wherein Prk represents the E. coli cells transformed with phosphoribulokinase gene (plasmid pYL25), Rubisco represents the E. coli cells transformed with Ribulose-1,5-bisphosphate carboxylase/oxygenase gene (plasmid pYL33), Prk+Rubisco represents the E. coli cells transformed with phosphoribulokinase and Ribulose-1,5-bisphosphate carboxylase/oxygenase genes (plasmid pYL35), and CT represents the E. coli cells transformed with the plasmid pET28a. FIG. 6A shows the growth of the transformed E. coli cells in the absence of IPTG; FIG. 6B shows the growth of the transformed E. coli cells in the presence of 0.5 mM IPTG (for inducing the expression of an exogenous gene). The results show that in the absence of IPTG, all the transformed E. coli cells grew normally; while in the presence of 0.5 mM IPTG, (1) the E. coli cells transformed with phosphoribulokinase gene (plasmid pYL25) could not grow normally, indicating that in the E. coli strain, Ribulose-5-phosphate was converted into an unmetabolizable final product, Ribulose-1,5-bisphosphate, under the catalysis of phosphoribulokinase, resulting in that the strain could not grow normally and even die, thereby demonstrating the E. coli strain could express active phosphoribulokinase; (2) the E. coli cells transformed with phosphoribulokinase and Ribulose-1,5-bisphosphate carboxylase/oxygenase genes (plasmid pYL35) could grow normally, indicating that the E. coli strain could express active Ribulose-1,5-bisphosphate carboxylase/oxygenase, which further converted the unmetabolizable Ribulose-1,5-bisphosphate into metabolizable glycerate 3-phosphate, thereby rescuing the lethal phenotype of the E. coli cells transformed with phosphoribulokinase gene (plasmid pYL25).

### Information of Sequence Listing:

SEQ ID NO: 1: GenBank: AP012278.1, the
nucleotide sequence of prk gene from
Synechocystis sp. PCC6803
ATGACCACACAGCTAGACCGCGTGGTTCTTATTGGTGTTGCCGGGGATTC
CGGTTGCGGTAAGTCTACTTTCTTACGTCGTTTAACGGATTTATTCGGCG
AAGAGTTCATGACGGTAATTTGTTTGGACGATTACCATAGTTTGGATCGC
CAGGGTAGAAAAGCCGCTGGGGTCACCGCCCTGGATCCCAGAGCCAACAA
TTTTGACCTCATGTATGAGCAGATTAAAACGCTCAAAAGTGGTCAATCCA
TTATGAAACCCATTTACAACCACGAAACGGGGCTGCTGGATCCGCCGGAA
AAAGTTGAACCCAACAAAGTGGTGGTTATTGAGGGTTTGCATCCCCTCTA
CGATGAACGGGTGCGGGAACTGGTGGATTTCGGGGTCTACCTGGACATCA
GCGAAGAAGTGAAAATTAACTGGAAAATTCAACGGGACATGGCCGAACGG
GGCCACACCTATGAAGATATTTTGGCTTCCATCAACGCCCGTAAGCCTGA
CTTCACTGCCTATATCGAGCCCCAAAAGCAATATGCGGACGTGGTGATCC
AGGTGTTGCCCACCCGCTTGATTGAGGACAAGGAAAGTAAACTCCTGCGG
GTTCGTCTTGTGCAAAAAGAAGGGGTTAAATTCTTCGAGCCAGCCTACCT
GTTTGACGAAGGTTCCACCATTGATTGGCGTCCCTGTGGTCGGAAGCTGA
CCTGTACCTATCCTGGCATCAAGATGTACTACGGCCCCGATAATTTTATG
GGCAACGAAGTATCTTTGCTGGAAGTGGACGGCAGGTTTGAAAACCTAGA
GGAAATGGTTTATGTGGAAAACCACCTCAGCAAGACTGGTACTAAGTACT
ACGGTGAAATGACCGAGTTGTTGCTCAAGCATAAGGATTACCCAGGCACT
GACAATGGTACTGGCCTGTTCCAGGTGTTAGTGGGTCTGAAAATGCGGGA
AGTTTACGAACAGTTAACGGCGGAAGCTAAGGTCCCGGCCTCTGTGTAA
SEQ ID NO: 2: GenBank: AP012278.1, the
nucleotide sequence of Rubisco gene
from Synechocystis sp. PCC6803
ATGGTACAAGCCAAAGCAGGGTTTAAGGCGGGCGTACAAGATTATCGCCT
GACCTACTATACCCCCGACTACACCCCCAAGGATACCGACCTGCTCGCCT
GCTTCCGTATGACCCCCCAACCGGGTGTACCTGCTGAAGAAGCCGCTGCT
GCGGTGGCCGCTGAGTCTTCCACCGGTACCTGGACCACCGTTTGGACTGA
CAACCTAACTGACTTGGACCGCTACAAAGGTCGTTGCTATGACCTGGAAG
CTGTTCCCAACGAAGATAACCAATATTTTGCTTTTATTGCCTATCCTCTA
GATTTATTTGAAGAAGGTTCCGTCACCAACGTTTTAACCTCTTTGGTCGG
TAACGTATTTGGTTTTAAGGCTCTGCGGGCCCTCCGTTTAGAAGATATTC
GTTTTCCCGTTGCTTTAATTAAAACCTTCCAAGGCCCTCCCCACGGTATT
ACCGTTGAGCGGGACAAATTAAACAAATACGGTCGTCCTCTGCTTGGTTG
TACCATCAAACCCAAACTTGGTCTGTCCGCCAAGAACTACGGTCGGGCTG
TTTACGAATGTCTCCGGGGTGGTTTGGACTTCACCAAAGACGACGAAAAC
ATCAACTCCCAGCCCTTCATGCGTTGGCGCGATCGTTTCCTCTTCGTTCA
AGAGGCGATCGAAAAAGCCCAGGCTGAGACCAACGAAATGAAAGGTCACT
ACCTGAACGTCACCGCTGGCACCTGCGAAGAAATGATGAAACGGGCCGAG
TTTGCCAAGGAAATTGGCACCCCCATCATCATGCATGACTTCTTCACCGG
CGGTTTCACTGCCAACACCACCCTCGCTCGTTGGTGTCGGGACAACGGCA
TTTTGCTCCATATTCACCGGGCAATGCACGCCGTAGTTGACCGTCAGAAA
AACCACGGGATCCACTTCCGGGTTTTGGCCAAGTGTCTGCGTCTGTCCGG
CGGTGACCACCTCCACTCCGGTACCGTGGTTGGTAAATTGGAAGGGGAAC
GGGGTATCACCATGGGCTTCGTTGACCTCATGCGCGAAGATTACGTTGAG
GAAGATCGCTCCCGGGGTATTTTCTTCACCCAAGACTATGCCTCCATGCC
TGGCACCATGCCCGTAGCTTCCGGTGGTATCCACGTATGGCACATGCCCG
CGTTGGTGGAAATCTTCGGTGATGATTCCTGCTTACAGTTTGGTGGTGGT
ACTTTGGGTCACCCCTGGGGTAATGCTCCCGGTGCAACCGCTAACCGTGT
TGCTTTGGAAGCTTGTGTTCAAGCTCGGAACGAAGGTCGTAACCTGGCTC
GCGAAGGTAATGACGTTATCCGGGAAGCCTGTCGTTGGTCCCCTGAGTTG
GCCGCCGCCTGCGAACTCTGGAAAGAGATCAAGTTTGAGTTCGAGGCCAT
GGATACCCTCTAAACCGGTGTTTGGATTGTCGGAGTTGTACTCGTCCGTT
AAGGATGAACAGTTCTTCGGGGTTGAGTCTGCTAACTAATTAGCCATTAA
CAGCGGCTTAACTAACAGTTAGTCATTGGCAATTGTCAAAAAATTGTTAA
TCAGCCAAAACCCACTGCTTACTGATGTTCAACTTCGACAGCAATTTACC
AATTACCGGGTAGAGTGTTCATGCAAACTAAGCACATAGCTCAGGCAACA
GTGAAAGTACTGCAAAGTTACCTCACCTACCAAGCCGTTCTCAGGATCCA
GAGTGAACTCGGGGAAACCAACCCTCCCCAGGCCATTTGGTTAAACCAGT
ATTTAGCCAGTCACAGTATTCAAAATGGAGAAACGTTTTTGACGGAACTC
CTGGATGAAAATAAAGAACTGGTACTCAGGATCCTGGCGGTAAGGGAAGA
CATTGCCGAATCAGTGTTAGATTTTTTGCCCGGTATGACCCGGAATAGCT
TAGCGGAATCTAACATCGCCCACCGCCGCCATTTGCTTGAACGTCTGACC
CGTACCGTAGCCGAAGTCGATAATTTCCCTTCGGAAACCTCCAACGGAGA
ATCAAACAACAACGATTCTCCCCCGTCCTAACGTAGTCATCAGCAAGGAA
AACTTTTAAATCGATGAAAACTTTACCCAAAGAGCGCCGCTACGAAACCC
TTTCTTACCTGCCCCCTTTAACCGATCAACAGATTGCTAAACAGGTTGAG
TTTCTGTTAGACCAGGGCTTTATTCCCGGCGTGGAATTTGAAGAAGACCC
CCAACCCGAAACCCACTTCTGGACCATGTGGAAACTGCCCTTCTTTGGTG
GTGCCACTGCCAACGAAGTTCTAGCCGAAGTACGGGAATGTCGTTCTGAG
AATCCCAACTGCTACATTCGGGTGATTGGTTTCGACAATATCAAACAGTG
CCAGACTGTAAGCTTTATTGTCCACAAACCCAACCAAAACCAAGGCCGTT
ACTAA
SEQ ID NO: 3: the sequence of primer PrkF
GGCATATGACCACACAGCTAGACCG
SEQ ID NO: 4: the sequence of primer PrkR
AGCTCGAGTTACACAGAGGCCGGGAC
SEQ ID NO: 5: the sequence of primer RubiscoF
GTTGTCGACGAAGGAGATATACATATGGTACAAGCCAAAGCAG
SEQ ID NO: 6: the sequence of primer RubiscoR
GACTCGAGACTGTAACTTGGGTAACGGCCTTGGT
SEQ ID NO: 7: GenBank: NP_441778.1, the amino
acid sequence encoded by prk gene from
Synechocystis sp. PCC6803
MTTQLDRVVLIGVAGDSGCGKSTFLRRLTDLFGEEFMTVICLDDYHSLDR
QGRKAAGVTALDPRANNFDLMYEQIKTLKSGQSIMKPIYNHETGLLDPPE
KVEPNKVVVIEGLHPLYDERVRELVDFGVYLDISEEVKINWKIQRDMAER
GHTYEDILASINARKPDFTAYIEPQKQYADVVIQVLPTRLIEDKESKLLR
VRLVQKEGVKFFEPAYLFDEGSTIDWRPCGRKLTCTYPGIKMYYGPDNFM
GNEVSLLEVDGRFENLEEMVYVENHLSKTGTKYYGEMTELLLKHKDYPGT
DNGTGLFQVLVGLKMREVYEQLTAEAKVPASV
SEQ ID NO: 8: GenBank: NP_442120.1, the amino
acid sequence of rbcL subunit encoded by
Rubisco gene from Synechocystis sp. PCC6803
MVQAKAGFKAGVQDYRLTYYTPDYTPKDTDLLACFRMTPQPGVPAEEAAA
AVAAESSTGTWTTVWTDNLTDLDRYKGRCYDLEAVPNEDNQYFAFIAYPL
DLFEEGSVTNVLTSLVGNVFGFKALRALRLEDIRFPVALIKTFQGPPHGI
TVERDKLNKYGRPLLGCTIKPKLGLSAKNYGRAVYECLRGGLDFTKDDEN
INSQPFMRWRDRFLFVQEAIEKAQAETNEMKGHYLNVTAGTCEEMMKRAE
FAKEIGTPIIMHDFFTGGFTANTTLARWCRDNGILLHIHRAMHAVVDRQK
NHGIHFRVLAKCLRLSGGDHLHSGTVVGKLEGERGITMGFVDLMREDYVE
EDRSRGIFFTQDYASMPGTMPVASGGIHVWHMPALVEIFGDDSCLQFGGG
TLGHPWGNAPGATANRVALEACVQARNEGRNLAREGNDVIREACRWSPEL
AAACELWKEIKFEFEAMDTL
SEQ ID NO: 9: GenBank: NP_442121.1, the amino
acid sequence of rbcX subunit encoded by
Rubisco gene from Synechocystis sp. PCC6803
VFMQTKHIAQATVKVLQSYLTYQAVLRIQSELGETNPPQAIWLNQYLASH
SIQNGETFLTELLDENKELVLRILAVREDIAESVLDFLPGMTRNSLAESN
IAHRRHLLERLTRTVAEVDNFPSETSNGESNNNDSPPS
SEQ ID NO: 10: GenBank: NP_442122.1, the
amino acid sequence of rbcS subunit
encoded by Rubisco gene from
Synechocystis sp. PCC6803
MKTLPKERRYETLSYLPPLTDQQIAKQVEFLLDQGFIPGVEFEEDPQPET
HFWTMWKLPFFGGATANEVLAEVRECRSENPNCYIRVIGFDNIKQCQTVS
FIVHKPNQNQGRY

### Deposition Information of the Samples of Biological Materials

The E. coli strain E2 as mentioned in the present invention was deposited by Qingdao Institute of Bioenergy and Bioprocess Technology, Chinese Academy of Sciences (CAS), (Songling Road No. 189, Laoshan District, Qingdao, P.R.China) in China General Microbiological Culture Collection Center (CGMCC) (Address: Institute of Microbiology, Chinese Academy of Sciences (CAS), No. 1 West Beichen Road, Chaoyang District, Beijing, China), under an Accession Number of CGMCC No. 5435 on Nov. 3, 2011.

### Examples

Certain embodiments of the present invention are illustrated in detail in combination with examples as follows. It is understood by those skilled in the art that the examples are used only for the purpose of illustrating the present invention, rather than limiting the protection scope of the present invention.

Unless indicated otherwise, the molecular biological experimental methods and immunological assays used in the present invention are carried out substantially in accordance with the methods as described in Sambrook J et al., Molecular Cloning: A Laboratory Manual (Second Edition), Cold Spring Harbor Laboratory Press, 1989, and F. M. Ausubel et al., Short Protocols in Molecular Biology, 3^{rd} Edition, John Wiley & Sons, Inc., 1995. The restriction enzymes are used under the conditions recommended by the product manufacturer. The reagents and instruments used in the present invention without marking out manufacturers are all conventional products commercially available from markets. Those skilled in the art understand that the examples are used for illustrating the present invention, but not intended to limit the protection scope of the present invention.

### Example 1

### Construction of Vectors and Strains for the Expression of phosphoribulokinase and Ribulose-1,5-bisphosphate carboxylase/oxygenase

In the Example, vectors and strains for the expression of phosphoribulokinase and Ribulose-1,5-bisphosphate carboxylase/oxygenase were constructed.

1. Construction of the Vector pYL25

The PCR amplification was performed using the genomic DNA of Synechocystis sp. PCC6803 as a template and using Prk-F (5′-GGC ATA TGA CCA CAC AGC TAG ACC G-3′) and Prk-R (5′-AGC TCG AGT TAC ACA GAG GCC GGG AC-3′) as primers. The product of PCR amplification then was cloned into pMD18-T vector (Takara, Catalog No.: D101A) according to the instructions of the manufacturer to obtain the vector pYL22. After verification of the vector pYL22 by sequencing, the vector pYL22 was digested by using NdeI (Takara, Catalog No.: D1161A) and XhoI (Takara, Catalog No.: D1073A), and a fragment of 1.7 kb was recovered. In addition, the plasmid pET28a (Novagen, Catalog NO.: 69864-3) was digested by using NdeI (Takara, Catalog No.: D1161A) and XhoI (Takara, Catalog No.: D1073A), and the fragment of 5.3 kb (which comprises a resistance gene) was recovered. The fragment of 1.7 kb and the fragment of 5.3 kb as obtained above were ligated by a ligase to produce the plasmid pYL25. The basic structure of the plasmid pYL25 was shown in FIG. 2, wherein P_{T7} represents T7 promoter, prk represents phosphoribulokinase, and Kan^{r} represents kanamycin resistance gene.

2. Construction of the Vector pYL33

The PCR amplification was performed using the genomic DNA of Synechocystis sp. PCC6803 as a template and using Rubisco-F (5′-AAC TCG AGG AAG GAG ATA ATG GTA CAA GCC AAA GCA G-3′) and Rubisco-R (5′-TGA CTC GAG ACT GTA CCT TAG TAA CGG CC-3′) as primers. The product of PCR amplification then was cloned into pMD18-T vector (Takara, Catalog No.: D101A) according to the instructions of the manufacturer to obtain the plasmid pYL30. After verification of the plasmid pYL30 by sequencing, the plasmid pYL30 was digested by using two enzymes, i.e. NdeI (Takara, Catalog No.: D1161A) and XhoI (Takara, Catalog No.: D1073A), and a fragment of 2.4 kb was recovered. In addition, the plasmid pET28a (Novagen) was digested by using NdeI (Takara, Catalog No.: D1161A) and XhoI (Takara, Catalog No.: D1073A), and the fragment of 5.3 kb (which comprises a resistance gene) was recovered. The fragment of 2.4 kb and the fragment of 5.3 kb as obtained above were ligated by a ligase to produce the plasmid pYL33. The basic structure of the plasmid pYL33 was shown in FIG. 3, wherein P_{T7} represents T7 promoter, Rubisco represents Ribulose-1,5-bisphosphate carboxylase/oxygenase, and Kan^{r} represents kanamycin resistance gene.

3. Construction of the Vector pYL35

The plasmid pYL33 was digested by using two enzymes, i.e. Sall (Takara, Catalog No.: D1080A) and XhoI (Takara, Catalog No.: D1073A), and a fragment of 2.4 kb (Rubisco gene) was recovered. In addition, the plasmid pYL25 was digested by using a single enzyme, XhoI (Takara, Catalog No.: D1073A), and a fragment of 7 kb (which comprises a promoter, Prk gene, a resistance gene, and His tags) was recovered. The fragment of 2.4 kb and the fragment of 7 kb as obtained above were ligated by a ligase to produce the plasmid pYL35. The basic structure of the plasmid pYL35 was shown in FIG. 4, wherein P_{T7} represents T7 promoter, prk represents phosphoribulokinase, Rubisco represents Ribulose-1,5-bisphosphate carboxylase/oxygenase, and Kan^{r} represents kanamycin resistance gene. The vector pET28a itself carried 2 His tags. After the construction procedure as described above, in the resultant plasmid pYL35, one His tag was fused to the N-terminal of the sequence of Prk gene and the other His tag was fused to the C-terminal of the sequence of Rubisco gene. Therefore, after introducing the plasmid pYL35 into a host cell, the host cell would express a Prk protein, the N-terminal of which a His tag was fused to, and a Rubisco protein, the C-terminal of which a His tag was fused to. The His tags might be useful in the detection and purification of the Prk protein and Rubisco protein expressed in the host cell.

4. Construction of Genetically Engineered Strains E1 and E2

pET28a (Novagen) and pYL35 were transformed into E. coli strain BL21 (DE3) by means of chemical transformation, respectively, to obtain the genetically engineered strains E1 (negative control) and E2. The construction of the strains was described in brief as followed.

1) E. coli from the strain stock preserved in glycerin was inoculated onto LB solid medium plate, and was subjected to inverted culture at 37 degrees C. overnight. Then, single colonies of a diameter of 2-3 mm were inoculated to a conical flask with 50 ml LB liquid medium, and were cultured at 37 degrees C. under shaking for 2 h (rotation speed 250 r/min). When OD500 reached about 0.4, 1.4 ml bacterial culture was drawn into an EP tube and was centrifugated at 7000 g for 2 min, and the supernatant was discarded. Centrifugation was carried out again at 7000 g for 2 min, and the supernatant was discarded. Then the cell pellet was suspended in 1 ml pre-cooled 0.1 mol/L CaCl₂ solution, and was incubated in ice bath for 10 min. After incubation in ice bath, the resultant suspension was centrifugated at 7000 g for 2 min, the supernatant was discarded and the bacterial pellet was collected.

2) The bacterial pellet was re-suspended in 200 μl pre-cooled 0.1 mol/L CaCl₂ solution, and was incubated in ice bath for 30 min. Plasmid DNA (50 ng/10 μl) was then added to the bacterial suspension, the resultant mixture was mixed gently and was incubated in ice bath for 20 min. After incubation in ice bath, the bacterial suspension was incubated in a water bath of 42 degrees C. for 2 min (without shaking), and was then immediately transferred to ice bath and kept standing for 2 min. After incubation in the ice bath, 800 μl LB liquid medium was added and the resultant mixture was cultured at 37 degrees C. for 45 min to facilitate the transformed E. coli strain to express the resistance gene.

3) The E. coli strain obtained in step 2) was inoculated onto LB solid medium plate comprising 50 μg/ml kanamycin, and was incubated at 37 degrees C. in a thermostatic incubator overnight. Single bacterial colonies in the plate were selected, and the transformants of interest were obtained after the presence of exogenous genes was verified by plasmid extraction or PCR identification.

### Example 2

### Verification of the Expression of Phosphoribulokinase and Ribulose-1,5-Bisphosphate Carboxylase/Oxygenase in Genetically Engineered Strains

In the Example, the expression of phosphoribulokinase and Ribulose-1,5-bisphosphate carboxylase/oxygenase in genetically engineered strains was verified by Western blot assay.

1. Extraction of Total Protein

The plasmid pYL35 was transformed into E. coli strain BL21 (DE3) by the method as described in Example 1, and the transformed E. coli strain was cultured at 16 degrees C. in 200 ml LB medium (comprising 0.5 mM IPTG, for inducing the expression of exogenous genes) overnight under shaking (rotation speed 200 r/min). Then, the bacterial cultures were centrifugated at 8000 rpm for 5 min, and the bacterial pellet was collected and the culture medium was discarded. 3 ml 4 degrees C. pre-cooled PBS (0.01M, pH7.2-7.3) was added to the bacterial pellet, the resultant mixture was shaken slightly for 1 min to wash the cells, and then the washing solution was discarded by centrifugation. The washing step was repeated twice (i.e. washing cells for three times in total) to remove the residual culture medium. Then, after the addition of 3 ml PBS, the bacterial cells was broken by sonication, and 1 ml was transferred into a centrifugal tube. The broken cells were centrifugated at 4 degrees C., 12000 rpm for 5 min. The supernatant (P+R)₂ and the precipitates (P+R)₁ were separated, and were transferred into new centrifugal tubes, respectively. 1 ml 1×Loading buffer (BioChip, CatalogNo.:370009-S2) was added to the precipitates (P+R)₁, the resultant mixture was mixed thoroughly and was boiled at 100 degrees C. for 5 min to finish the preparation of the sample, and then 6 μl was taken for loading. In addition, 6 μl supernatant (P+R)₂ was taken, to which 3 μl 5× Loading buffer (BioChip, CatalogNo.:370009-S2) was added, and water was added until the volume reached 15 μl (the final concentration of the Loading buffer was 1×). Then, the result mixture was boiled at 100 degrees C. for 5 min to finish the preparation of the sample for loading.

2. SDS-PAGE Electrophoresis and Transferring to a Membrane

12% polyacrylamide gel was used for SDS-PAGE electrophoresis (electrophoresis was performed for 4-5 h at a voltage of 40V or 60V), and after electrophoresis, the protein sample separated in the gel was transferred to a nitrocellulose membrane.

3. Membrane Staining

After transferring to a nitrocellulose membrane, the membrane was stained with 1×ponceau staining solution for 5 min in a shaker, and then was washed with water to remove the residual staining solution. After staining, protein bands were observed in the membrane. The membrane was dried in the air for further use.

4. Immunoassay 1) After soaking the membrane in TBS (8.8 g NaCl, 1M Tris (PH8.0), metered to a final volume of 1 L), the membrane was transferred to a plate comprising a blocking buffer (5% skim milk powder, in TBST (8.8 g NaCl, 1M Tris (PH8.0), 0.5 ml Tween20, metered to a final volume of 1 L)), and was blocked at room temperature in a shaker for 1 h.

2) After blocking, mouse anti-His tag antibody (Invitrogen, Catalog No.: 37-2900, which was 1:10000 diluted with TBST) was used to incubated the membrane at room temperature for 1-2 h; TBST was then used to wash the membrane at room temperature in the shaker twice, each for 10 min; then, the membrane was washed with TBS once for 10 min.

3) Goat anti-mouse IgG-Alkaline Phosphatase (Invitrogen, Catalog No.: G-21060, which was 1:3000 diluted with TBST) was used to incubated the membrane at room temperature for 1-2 h; TBST was then used to wash the membrane at room temperature in the shaker twice, each for 10 min; then, the membrane was washed with TBS once for 10 min.

4) According to the manufacturer's instruction, NBT-BCIP Kit (Roche, Catalog No.:11681451001) was used to develop the membrane. The results of Western Blot assay were shown in FIG. 5. The results showed that both phosphoribulokinase and Ribulose-1,5-bisphosphate carboxylase/oxygenase were expressed in the strains transformed with the plasmid pYL35.

### Example 3

### Verification of Activities of Phosphoribulokinase and Ribulose-1,5-Bisphosphate Carboxylase/Oxygenase Expressed in Genetically Engineered Strains

In the Example, activities of phosphoribulokinase and Ribulose-1,5-bisphosphate carboxylase/oxygenase expressed in genetically engineered strain E2 were verified. The following experiment was carried out to verify that the plasmids constructed in the present invention could express active phosphoribulokinase and Ribulose-1,5-bisphosphate carboxylase/oxygenase in E. coli strains. The plasmid pYL25, the plasmid pYL33, the plasmids pYL35 and pET28a were transformed into E. coli strains, respectively. Then, the transformed E. coli strains were plated to a LB solid medium plate containing 0 mM IPTG and 50 μg mL⁻¹ kanamycin (FIG. 6A) and a LB solid medium plate containing 0.5 mM IPTG and 50 μg mL⁻¹ kanamycin (FIG. 6B), respectively, and were cultured overnight at 37 degrees C. The growth of E. coli strains was observed, and the observed results were shown in FIG. 6.

1. Verification of Phosphoribulokinase Activity

Phosphoribulokinase catalyzed the conversion of Ribulose-5-phosphate into Ribulose-1,5-bisphosphate. In a heterotrophic microorganism, E. coli, Ribulose-5-phosphate was an important reaction substrate in PPP pathway (pentose phosphate pathway), while Ribulose-1,5-bisphosphate was an unmetabolizable final product. Therefore, when phosphoribulokinase was expressed in E. coli at a large level, it would compete with the PPP pathway for the important reaction substrate Ribulose-5-phosphate and the unmetabolizable final product, Ribulose-1,5-bisphosphate, would be accumulated in a large amount, resulting in that the E. coli strains overexpressing phosphoribulokinase could not grow normally and the lethal phenotype occurred.

The results in FIG. 6A showed that in the absence of IPTG (i.e. without inducing expression of exogenous genes), all the transformed E. coli strains could grow normally in the LB solid medium plate containing kanamycin. This showed that the plasmids of interest were transformed into E. coli, and the kanamycin resistance gene was expressed. Meanwhile, due to the absence of IPTG, the E. coli strains did not express the Prk gene and/or Rubisco gene comprised in the plasmids, and therefore the strains grew normally and did not die.

Furthermore, the results in FIG. 6B showed that in the presence of 0.5 mM IPTG (i.e. for inducing expression of exogenous genes), the E. coli strain transformed with plasmid pET28a grew normally in the LB solid medium plate containing kanamycin, while the E. coli strain transformed with plasmid pYL25 could not grow. This was consistent with the results as expected, i.e. expression of phosphoribulokinase at high level in the E. coli strain would result in abnormal growth of the E. coli strain and death. Thus, the results (in particular, by comparing the regions indicated by Prk in FIG. 6A and FIG. 6B) showed that the E. coli strain transformed with plasmid pYL25 expressed active phosphoribulokinase when driven by P_{T7} promoter and induced by IPTG.

2. Verification of Ribulose-1,5-Bisphosphate Carboxylase/Oxygenase Activity

Ribulose-1,5-bisphosphate carboxylase/oxygenase catalyzed the conversion of Ribulose-1,5-bisphosphate into glycerate 3-phosphate. Therefore, when Ribulose-1,5-bisphosphate carboxylase/oxygenase was correctly expressed, it would form a metabolic pathway with phosphoribulokinase to further convert Ribulose-1,5-bisphosphate, which could not be metabolized in E. coli, into glycerate 3-phosphate, which could be further metabolized, thereby rescuing the lethal phenotype of cells caused by overproduction of Ribulose-1,5-bisphosphate (see FIG. 1C).

The results in FIG. 6B showed that in the presence of 0.5 mM IPTG (i.e. for inducing expression of exogenous genes), the E. coli strain transformed with the plasmid pYL33 grew normally in the LB solid medium plate containing kanamycin. The results (in particular, by comparing the regions indicated by Rubisco in FIG. 6A and FIG. 6B) that expression of Rubisco alone did not bring about significant adverse effect to the growth of the E. coli strain.

Furthermore, the results in FIG. 6B showed that in the presence of 0.5 mM IPTG (i.e. for inducing expression of exogenous genes), the E. coli strain transformed with the plasmid pYL35 grew normally in the LB solid medium plate containing kanamycin. The results (in particular, by comparing the region indicated by Prk and the region indicated by Prk+Rubisco in FIG. 6B) showed that the E. coli strain transformed with plasmid pYL35 (comprising phosphoribulokinase and Ribulose-1,5-bisphosphate carboxylase/oxygenase genes) could express active Ribulose-1,5-bisphosphate carboxylase/oxygenase, which formed a carbon dioxide fixation pathway with phosphoribulokinase, thereby further converting the unmetabolizable Ribulose-1,5-bisphosphate into metabolizable glycerate 3-phosphate, thereby rescuing the lethal phenotype of the E. coli strain transformed with phosphoribulokinase gene alone (the plasmid pYL25).

### Example 4

### Detection of Carbon Dioxide Emission in Genetically Engineered E. coli Strains

1. Experimental Steps

(1) Culturing manner: shaking culture. A normal 250 mL conical flask with 100 mL liquid M9 medium (see J. Sambrook et al., Molecular Cloning: A Laboratory Manual, the second edition, Cold Spring Harbor Laboratory Press, 1989) was used. In the medium, 4 g/L glucose was the only carbon source, and 50 ug mL⁻¹ kanamycin was added. The medium was inoculated with the genetically engineered strain E1 (negative control) or E2 as constructed in Example 1, respectively. The initial inoculation concentration was OD₆₀₀ 0.05. The E. coli strains were cultured at 37 degrees C., 200 rpm until OD₆₀₀ reached 0.4-0.6. Then, 0.5 mM IPTG was added and the culturing was performed for further 25 h.

(2) 50 mL of culture solution was taken and was centrifugated at 8000 rpm for 5 min, and the bacterial pellet and the supernatant were collected, respectively. The bacterial pellet was suspended in 3 ml sugar-free M9 medium and blow washed for 1 min, and then was centrifugated at 8000 rpm for 5 min to remove the washing solution. The washing step was repeated twice (i.e. washing the cells for three times in total) to remove the residual glucose, and the bacterial pellet was collected after the last centrifugation. In addition, the supernatant as collected before was centrifugated at 12000 rpm for 10 min, and the supernatant recollected was filtrated, and then about 10 ml of the filtrated supernatant (i.e. the residual fermentation solution) was collected;

(3) The bacterial pellet was baking-dried and its dry weight was measured. According to the manufacturer's instructions, Total Carbon and Total Nitrogen Analyzer, Elementar liquid TOCII (German, Elementar Co.), was used to detect the carbon content (i.e. the inorganic and organic carbon content) in the residual fermentation solution, the carbon content in the washed bacterial pellet, and the carbon content in the initial medium.

(4) The total carbon dioxide emission (represented by the carbon content) and the carbon dioxide emission per OD₆₀₀ were calculated by the carbon content in the initial medium, the carbon content in the bacterial pellet, and the carbon content in the residual fermentation solution as follows:

Total carbon dioxide emission=Carbon content in the initial medium−Carbon content in the bacterial pellet−Carbon content in the residual fermentation solution;

Carbon dioxide emission per OD₆₀₀=Total carbon dioxide emission/OD₆₀₀ of Fermentation solution.

2. Experimental Results

The carbon content in the initial medium, the carbon content in the bacterial pellet, and the carbon content in the residual fermentation solution as measured, and the total carbon dioxide emission and the carbon dioxide emission per OD₆₀₀ as calculated, were shown in Table 1.

TABLE 1
Carbon metabolism distribution of E1 and E2

| | | | | Carbon content | Inorganic carbon | Organic carbon | | |
|---|---|---|---|---|---|---|---|---|
| | Carbon content in | | Dry weight | in bacterial | content in residual | content in residual | | |
| | initial medium | | of bacteria | pellet | fermentation solution | fermentation solution | CO₂ emission | |
| Strain | (mg/L) | OD_{600 nm} | (mg/L) | (mg/L) | (mg/L) | (mg/L) | (mg/L) | (mg/L/OD) |
| E1 | 1577 | 2.38 ± 0.03 | 581 ± 39 | 246 ± 15 | 13 ± 2 | 284 ± 6 | 1034 ± 23 | 434 ± 4 |
| E2 | 1577 | 2.94 ± 0.08 | 735 ± 36 | 333 ± 19 | 17 ± 3 | 408 ± 7 | 819 ± 21 | 279 ± 7 |

The results in Table 1 showed that as compared with the strain E1, the strain E2 produced more biomass (i.e. more cells were obtained) with less carbon consumption (more organic carbons were left in the residual fermentation solution), and significantly reduces the carbon dioxide emission during fermentation (the carbon emission per liter fermentation solution per OD₆₀₀ bacteria was reduced by 33%). The results showed that by expressing phosphoribulokinase and Ribulose-1,5-bisphosphate carboxylase/oxygenase in a heterotrophic microorganism (E. coli), the inventors successfully constructed a carbon dioxide fixation pathway in the heterotrophic microorganism (E. coli), and the constructed carbon dioxide fixation pathway effectively fixed carbon dioxide, thereby significantly reducing carbon dioxide emission during fermentation of the microorganisms and enhancing utilization rate of carbon source/energy.

Although the specific embodiments of the present invention have been described in details, those skilled in the art would understand that, according to the teachings disclosed in the specification, details can be modified and changed without departing from the sprit or scope of the present invention as generally described. The scope of the present invention is given by the appended claims and any equivalents thereof.

1. A microorganism comprising:
a first gene; and
a second gene;
wherein the first gene is selected from the group consisting of:
1) a phosphoribulokinase (Prk) gene (EC2.7.1.19);
2) a nucleotide sequence that has at least 80% identity to the sequence of a phosphoribulokinase (Prk) gene (EC2.7.1.19); and
3) a nucleotide sequence capable of hybridizing with the sequence of a phosphoribulokinase (Prk) gene (EC2.7.1.19) under stringent hybridization conditions; and
wherein the second gene is selected from the group consisting of:
4) a Ribulose-1,5-bisphosphate carboxylase/oxygenase (Rubisco) gene (EC 4.1.1.39);
5) a nucleotide sequence that has at least 80% identity to the sequences of a Ribulose-1,5-bisphosphate carboxylase/oxygenase (Rubisco) gene (EC 4.1.1.39); and
6) a nucleotide sequence capable of hybridizing with the sequences of a Ribulose-1,5-bisphosphate carboxylase/oxygenase (Rubisco) gene (EC 4.1.1.39) under stringent hybridization conditions;
wherein the microorganism is a heterophic microorganism.

2. The microorganism of claim 1, wherein the phosphoribulokinase (Prk) gene is derived from cyanobacteria or chlorella.

3. The microorganism of claim 1 wherein the phosphoribulokinase (Prk) gene encodes a polypeptide with an amino acid sequence as shown in SEQ ID NO: 7.

4. The microorganism of claim 3 wherein phosphoribulokinase (Prk) gene has the nucleic acid sequence as shown in SEQ ID NO: 1.

5. The microorganism of claim 1, wherein the Ribulose-1,5-bisphosphate carboxylase/oxygenase (Rubisco) gene is derived from cyanobacteria, chlorella, or plants.

6. The construct of claim 1 wherein the Ribulose-1,5-bisphosphate carboxylase/oxygenase (Rubisco) gene encodes three polypeptide subunits as shown in SEQ ID NOs: 8-10.

7. The microorganism of claim 6 wherein the Ribulose-1,5-bisphosphate carboxylase/oxygenase (Rubisco) gene has the sequence as shown in SEQ ID NO: 2.

8. The microorganism of claim 1, wherein the heterotrophic microorganism is selected from the group consisting of heterotrophic bacteria, fungus, and yeast.

9. The microorganism of claim 8 wherein the heterotrophic microorganism is selected from the group consisting of Saccharomyces cerevisiae, Pichia, Aspergillus niger, E. coli, Bacillus aceticus, Pseudomonas, Bacillus brevis, Corynebacterium, Bacillus subtilis, Bacillus stearothermophilus, Clostridium acetobutylicum, Clostridium butyricum, and Clostridium pasteurianum.

10. The microorganism of claim 9, wherein the heterotrophic microorganism is E. coli as deposited in China General Microbiological Culture Collection Center (CGMCC) under Accession Number of CGMCC No. 5435.

11. A method for fixing carbon dioxide in a heterotrophic microorganism or reducing carbon dioxide emission in a heterotrophic microorganism, comprising:
introducing a first gene and a second gene into a heterotrophic microorganism, wherein the first gene is selected from the group consisting of:
1) a phosphoribulokinase (Prk) gene (EC2.7.1.19);
2) a nucleotide sequence that has at least 80% identity to the sequence of a phosphoribulokinase (Prk) gene (EC2.7.1.19); and
3) a nucleotide sequence capable of hybridizing with the sequence of a phosphoribulokinase (Prk) gene (EC2.7.1.19) under stringent hybridization conditions; and
wherein the second gene is selected from the group consisting of:
4) a Ribulose-1,5-bisphosphate carboxylase/oxygenase (Rubisco) gene (EC 4.1.1.39);
5) a nucleotide sequence that has at least 80% identity to the sequences of a Ribulose-1,5-bisphosphate carboxylase/oxygenase (Rubisco) gene (EC 4.1.1.39); and
6) a nucleotide sequence capable of hybridizing with the sequences of a Ribulose-1,5-bisphosphate carboxylase/oxygenase (Rubisco) gene (EC 4.1.1.39) under stringent hybridization conditions; and
allowing the heterotrophic microorganism to express the first gene and the second gene.

12. The method of claim 11, wherein the phosphoribulokinase (Prk) gene is derived from cyanobacteria or chlorella.

13. The method of claim 12 wherein the phosphoribulokinase (Prk) gene encodes a protein as shown in SEQ ID NO: 7.

14. The method of claim 13 wherein the phosphoribulokinase (Prk) gene has the sequence as shown in SEQ ID NO: 1.

15. The method of claim 1, wherein the Ribulose-1,5-bisphosphate carboxylase/oxygenase (Rubisco) gene is derived from cyanobacteria, chlorella, or plants.

16. The method of claim 16 wherein the Ribulose-1,5-bisphosphate carboxylase/oxygenase (Rubisco) gene encodes three polypeptide subunits as shown in SEQ ID NOs: 8-10.

17. The method of claim 16 wherein the Ribulose-1,5-bisphosphate carboxylase/oxygenase (Rubisco) gene has the sequence as shown in SEQ ID NO: 2.

18. The method of claim 11, wherein at least one of the first gene or the second gene is introduced into the heterotrophic microorganism by one or more vectors.

19. The method of claim 11, wherein the first gene and the second gene are incorporated into the genome of the heterotrophic microorganism.

20. The method of claim 11, wherein the first gene and the second gene are present as episomes in the heterotrophic microorganism.

21. A vector comprising
a first gene; and
a second gene,
wherein the first gene is selected from the group consisting of:
1) a phosphoribulokinase (Prk) gene (EC2.7.1.19);
2) a nucleotide sequence that has at least 80% identity to the sequence of a phosphoribulokinase (Prk) gene (EC2.7.1.19); and
3) a nucleotide sequence capable of hybridizing with the sequence of a phosphoribulokinase (Prk) gene (EC2.7.1.19) under stringent hybridization conditions; and
wherein the second gene is selected from the group consisting of:
4) a Ribulose-1,5-bisphosphate carboxylase/oxygenase (Rubisco) gene (EC 4.1.1.39);
5) a nucleotide sequence that has at least 80% identity to the sequences of a Ribulose-1,5-bisphosphate carboxylase/oxygenase (Rubisco) gene (EC 4.1.1.39); and
6) a nucleotide sequence capable of hybridizing with the sequences of a Ribulose-1,5-bisphosphate carboxylase/oxygenase (Rubisco) gene (EC 4.1.1.39) under stringent hybridization conditions; and
an expression regulatory sequence operably linked to at least one of the first gene and the second gene.

22. The vector of claim 21 wherein the expression regulatory sequence is selected from the group consisting of a promoter, a terminator, and an enhancer.

23. The vector of claim 22, wherein the promoter is selected from the group consisting of T7 promoter, CMV promoter, pBAD promoter, Trc promoter, Tac promoter, and lacUV5 promoter.


SEQUENCE LISTING (10 sequences)

SEQ ID NO: 1 - DNA, 999 bp, Synechocystis sp. PCC6803
atgaccacacagctagaccgcgtggttcttattggtgttgccggggattccggttgcggtaagtctactttcttacgtcgtttaacggatttattcggcgaagagttcatgacggtaatttgtttggacgattaccatagtttggatcgccagggtagaaaagccgctggggtcaccgccctggatcccagagccaacaattttgacctcatgtatgagcagattaaaacgctcaaaagtggtcaatccattatgaaacccatttacaaccacgaaacggggctgctggatccgccggaaaaagttgaacccaacaaagtggtggttattgagggtttgcatcccctctacgatgaacgggtgcgggaactggtggatttcggggtctacctggacatcagcgaagaagtgaaaattaactggaaaattcaacgggacatggccgaacggggccacacctatgaagatattttggcttccatcaacgcccgtaagcctgacttcactgcctatatcgagccccaaaagcaatatgcggacgtggtgatccaggtgttgcccacccgcttgattgaggacaaggaaagtaaactcctgcgggttcgtcttgtgcaaaaagaaggggttaaattcttcgagccagcctacctgtttgacgaaggttccaccattgattggcgtccctgtggtcggaagctgacctgtacctatcctggcatcaagatgtactacggccccgataattttatgggcaacgaagtatctttgctggaagtggacggcaggtttgaaaacctagaggaaatggtttatgtggaaaaccacctcagcaagactggtactaagtactacggtgaaatgaccgagttgttgctcaagcataaggattacccaggcactgacaatggtactggcctgttccaggtgttagtgggtctgaaaatgcgggaagtttacgaacagttaacggcggaagctaaggtcccggcctctgtgtaa

SEQ ID NO: 2 - DNA, 2405 bp, Synechocystis sp. PCC6803
atggtacaagccaaagcagggtttaaggcgggcgtacaagattatcgcctgacctactatacccccgactacacccccaaggataccgacctgctcgcctgcttccgtatgaccccccaaccgggtgtacctgctgaagaagccgctgctgcggtggccgctgagtcttccaccggtacctggaccaccgtttggactgacaacctaactgacttggaccgctacaaaggtcgttgctatgacctggaagctgttcccaacgaagataaccaatattttgcttttattgcctatcctctagatttatttgaagaaggttccgtcaccaacgttttaacctctttggtcggtaacgtatttggttttaaggctctgcgggccctccgtttagaagatattcgttttcccgttgctttaattaaaaccttccaaggccctccccacggtattaccgttgagcgggacaaattaaacaaatacggtcgtcctctgcttggttgtaccatcaaacccaaacttggtctgtccgccaagaactacggtcgggctgtttacgaatgtctccggggtggtttggacttcaccaaagacgacgaaaacatcaactcccagcccttcatgcgttggcgcgatcgtttcctcttcgttcaagaggcgatcgaaaaagcccaggctgagaccaacgaaatgaaaggtcactacctgaacgtcaccgctggcacctgcgaagaaatgatgaaacgggccgagtttgccaaggaaattggcacccccatcatcatgcatgacttcttcaccggcggtttcactgccaacaccaccctcgctcgttggtgtcgggacaacggcattttgctccatattcaccgggcaatgcacgccgtagttgaccgtcagaaaaaccacgggatccacttccgggttttggccaagtgtctgcgtctgtccggcggtgaccacctccactccggtaccgtggttggtaaattggaaggggaacggggtatcaccatgggcttcgttgacctcatgcgcgaagattacgttgaggaagatcgctcccggggtattttcttcacccaagactatgcctccatgcctggcaccatgcccgtagcttccggtggtatccacgtatggcacatgcccgcgttggtggaaatcttcggtgatgattcctgcttacagtttggtggtggtactttgggtcacccctggggtaatgctcccggtgcaaccgctaaccgtgttgctttggaagcttgtgttcaagctcggaacgaaggtcgtaacctggctcgcgaaggtaatgacgttatccgggaagcctgtcgttggtcccctgagttggccgccgcctgcgaactctggaaagagatcaagtttgagttcgaggccatggataccctctaaaccggtgtttggattgtcggagttgtactcgtccgttaaggatgaacagttcttcggggttgagtctgctaactaattagccattaacagcggcttaactaacagttagtcattggcaattgtcaaaaaattgttaatcagccaaaacccactgcttactgatgttcaacttcgacagcaatttaccaattaccgggtagagtgttcatgcaaactaagcacatagctcaggcaacagtgaaagtactgcaaagttacctcacctaccaagccgttctcaggatccagagtgaactcggggaaaccaaccctccccaggccatttggttaaaccagtatttagccagtcacagtattcaaaatggagaaacgtttttgacggaactcctggatgaaaataaagaactggtactcaggatcctggcggtaagggaagacattgccgaatcagtgttagattttttgcccggtatgacccggaatagcttagcggaatctaacatcgcccaccgccgccatttgcttgaacgtctgacccgtaccgtagccgaagtcgataatttcccttcggaaacctccaacggagaatcaaacaacaacgattctcccccgtcctaacgtagtcatcagcaaggaaaacttttaaatcgatgaaaactttacccaaagagcgccgctacgaaaccctttcttacctgccccctttaaccgatcaacagattgctaaacaggttgagtttctgttagaccagggctttattcccggcgtggaatttgaagaagacccccaacccgaaacccacttctggaccatgtggaaactgcccttctttggtggtgccactgccaacgaagttctagccgaagtacgggaatgtcgttctgagaatcccaactgctacattcgggtgattggtttcgacaatatcaaacagtgccagactgtaagctttattgtccacaaacccaaccaaaaccaaggccgttactaa

SEQ ID NO: 3 - DNA, 25 bp, artificial
  primer
ggcatatgaccacacagctagaccg

SEQ ID NO: 4 - DNA, 26 bp, artificial
  primer
agctcgagttacacagaggccgggac

SEQ ID NO: 5 - DNA, 43 bp, artificial
  primer
gttgtcgacgaaggagatatacatatggtacaagccaaagcag

SEQ ID NO: 6 - DNA, 34 bp, artificial
  primer
gactcgagactgtaacttgggtaacggccttggt

SEQ ID NO: 7 - protein, 332 aa, Synechocystis sp. PCC6803
MTTQLDRVVLIGVAGDSGCGKSTFLRRLTDLFGEEFMTVICLDDYHSLDRQGRKAAGVTALDPRANNFDLMYEQIKTLKSGQSIMKPIYNHETGLLDPPEKVEPNKVVVIEGLHPLYDERVRELVDFGVYLDISEEVKINWKIQRDMAERGHTYEDILASINARKPDFTAYIEPQKQYADVVIQVLPTRLIEDKESKLLRVRLVQKEGVKFFEPAYLFDEGSTIDWRPCGRKLTCTYPGIKMYYGPDNFMGNEVSLLEVDGRFENLEEMVYVENHLSKTGTKYYGEMTELLLKHKDYPGTDNGTGLFQVLVGLKMREVYEQLTAEAKVPASV

SEQ ID NO: 8 - protein, 470 aa, Synechocystis sp. PCC6803
MVQAKAGFKAGVQDYRLTYYTPDYTPKDTDLLACFRMTPQPGVPAEEAAAAVAAESSTGTWTTVWTDNLTDLDRYKGRCYDLEAVPNEDNQYFAFIAYPLDLFEEGSVTNVLTSLVGNVFGFKALRALRLEDIRFPVALIKTFQGPPHGITVERDKLNKYGRPLLGCTIKPKLGLSAKNYGRAVYECLRGGLDFTKDDENINSQPFMRWRDRFLFVQEAIEKAQAETNEMKGHYLNVTAGTCEEMMKRAEFAKEIGTPIIMHDFFTGGFTANTTLARWCRDNGILLHIHRAMHAVVDRQKNHGIHFRVLAKCLRLSGGDHLHSGTVVGKLEGERGITMGFVDLMREDYVEEDRSRGIFFTQDYASMPGTMPVASGGIHVWHMPALVEIFGDDSCLQFGGGTLGHPWGNAPGATANRVALEACVQARNEGRNLAREGNDVIREACRWSPELAAACELWKEIKFEFEAMDTL

SEQ ID NO: 9 - protein, 138 aa, Synechocystis sp. PCC6803
VFMQTKHIAQATVKVLQSYLTYQAVLRIQSELGETNPPQAIWLNQYLASHSIQNGETFLTELLDENKELVLRILAVREDIAESVLDFLPGMTRNSLAESNIAHRRHLLERLTRTVAEVDNFPSETSNGESNNNDSPPS

SEQ ID NO: 10 - protein, 113 aa, Synechocystis sp. PCC6803
MKTLPKERRYETLSYLPPLTDQQIAKQVEFLLDQGFIPGVEFEEDPQPETHFWTMWKLPFFGGATANEVLAEVRECRSENPNCYIRVIGFDNIKQCQTVSFIVHKPNQNQGRY